(19) United States
(12) Patent Application Publication
(10) Pub. No.: US 2024/0077491 A1
(43) Pub. Date: Mar. 7, 2024

(54) METHOD AND SYSTEMS FOR IDENTIFYING A SEQUENCE OF MONOMER UNITS OF A BIOLOGICAL OR SYNTHETIC HETEROPOLYMER

(71) Applicant: Albert-Ludwigs-Universität Freiburg, Freiburg (DE)

(72) Inventors: Jan Behrends, Stegen (DE); Tobias Ensslen, Wannweil (DE)

(21) Appl. No.: 18/261,248

(22) PCT Filed: Jan. 18, 2022
(86) PCT No.: PCT/EP2022/050990

§ 371 Date: Jul. 12, 2023

(30) Foreign Application Priority Data
Jan. 18, 2021 (DE) 102021200425.3

(51) Int. Cl. G01N 33/68

(52) U.S. Cl. CPC G01N 33/6824; G01N 2333/96433

(57) ABSTRACT

The present invention relates to a method for the identification a sequence of monomer building blocks of a biological or synthetic heteropolymer. The invention also relates to the use of a nanopore for identifying a sequence of monomer building blocks of a biological or synthetic heteropolymer. The invention further relates to a computer-implemented method, computer program code, and data processing system for identifying a sequence of monomer building blocks of a biological or synthetic heteropolymer.

The present invention relates to a method for identifying a sequence of monomer building blocks of a biological or synthetic heteropolymer. The invention also relates to the use of a nanopore for identifying a sequence of monomer building blocks of a biological or synthetic heteropolymer. The invention further relates to a computer-implemented method, computer program code, and data processing system for identifying a sequence of monomer building blocks of a biological or synthetic heteropolymer.

In recent decades, considerable progress has been made in technologies for extracting genetic information from cells and tissues, including next-generation single-molecule nucleic acid sequencing techniques. In contrast, similar development for direct identification, discrimination, and sequencing of proteins from cellular or acellular samples has yet to occur. While DNA and RNA sequences provide some prediction of the proteins expressed in a cell or tissue, direct determination of the proteome, e.g., from tumor cells, is more relevant for elucidating biological properties. Indeed, in situations where the presence of specific proteins or protein isoforms is desired or, as the case may be, undesired, such as in vitro protein synthesis for biologicals or biosimilars, per se protein detection and identification is required.

The identification of proteins in complex mixtures currently relies on mass spectrometry of ionized molecules in the gas phase, a powerful but costly technology that requires large equipment. The present invention consists in a novel approach combining highly controlled and automated, preferably enzymatic, fragmentation, using both sequence-specific endopeptidases and exopeptidases, with a newly developed principle of “peptide spectrometry through nanopores” for purposes of label-free characterization of protein mixtures, including identification, discrimination and ultimately protein sequencing.

Nanopore size spectroscopy was first demonstrated for synthetic polymers, but has recently been shown to be applicable to peptides, enabling their highly sensitive, label-free discrimination (Piguet et al. 2018; Ouldali et al. 2020). Importantly, this technique is able to detect differences in individual amino acid residues and, unlike mass spectrometry, distinguish between peptides of the same mass, e.g., peptides containing either the stereoisomers leucine or isoleucine (Ouldali et al. 2020), or characterized by sequence isomerism.

The current standard method for identifying proteins from mixtures involves a series of separation steps, such as liquid chromatography or (2D) gel electrophoresis, followed by tryptic digestion to peptide fragments, and mass spectrometry, e.g. electrospray ionization (ESI), or matrix-assisted laser desorption/ionization (MALDI), followed by separation according to time-of-flight (TOF), or in a quadru-(Q)/multipole field and subsequent correlation with known proteins in databases. Mass spectrometry, although a powerful technique, requires costly and bulky equipment and has significant shortcomings in terms of detection limits and dynamic sensitivity range. A more fundamental drawback is that peptides of the same mass but different composition (e.g., containing leucine or isoleucine) cannot be distinguished without derivatization. For these reasons, novel solutions are needed to identify, distinguish, and ultimately sequence proteins with single-molecule sensitivity.

In contrast to nanopore-mediated single-molecule DNA sequencing, where only 4 nucleobases of the same charge need to be distinguished, in the case of protein structure elucidation, the problem is incomparably more complex by comparison because of the 20 proteinogenic amino acids (aa). To date, this field is still in its infancy, but some progress has already been made, which is summarized below.

Single molecule detection through nanopores is based on analyzing the reduction in electrical conductivity that occurs when an analyte, e.g., a DNA strand or a peptide, diffuses or migrates into a molecularly sized water-filled channel located in an insulator, i.e., a nanopore. The principle of electrical detection of the transport of molecules through a nanopore, which may be a protein channel or an artificial channel, e.g., a nanoscale aperture in a solid membrane or a nanotube or DNA origami structure inserted into a lipid membrane or a nanoscale hole inserted into a solid membrane, is well known. The membrane is subjected to a potential difference that induces an ionic current through the nanopore in the presence of an electrolyte solution or other ionically conductive medium (e.g., an ionic liquid). The interaction of a molecule with the channel of a nanopore, in particular the entry of the molecule into the channel, the presence of the molecule in the channel, or the passage of the molecule through the channel, thereby induces a measurable decrease in the current, provided that the conductive medium in the channel has a higher electrical conductivity than the analyte and vice versa.

Biological (protein) nanopores forming such channels through insulating lipid bilayers were the first nanopores shown to be capable of detecting single molecules, and they enable current nanopore-based DNA sequencing techniques. Alternatively, nanoscopic pores can be fabricated by various drilling or etching techniques in solid-state materials such as thin SiN membranes. These solid-state nanopores are promising, although fabricating solid-state nanopores that are as identical as possible is a technical challenge. In contrast, pore-forming proteins are constructed with atomic precision and have evolved over millions of years to enable solute transport across membranes.

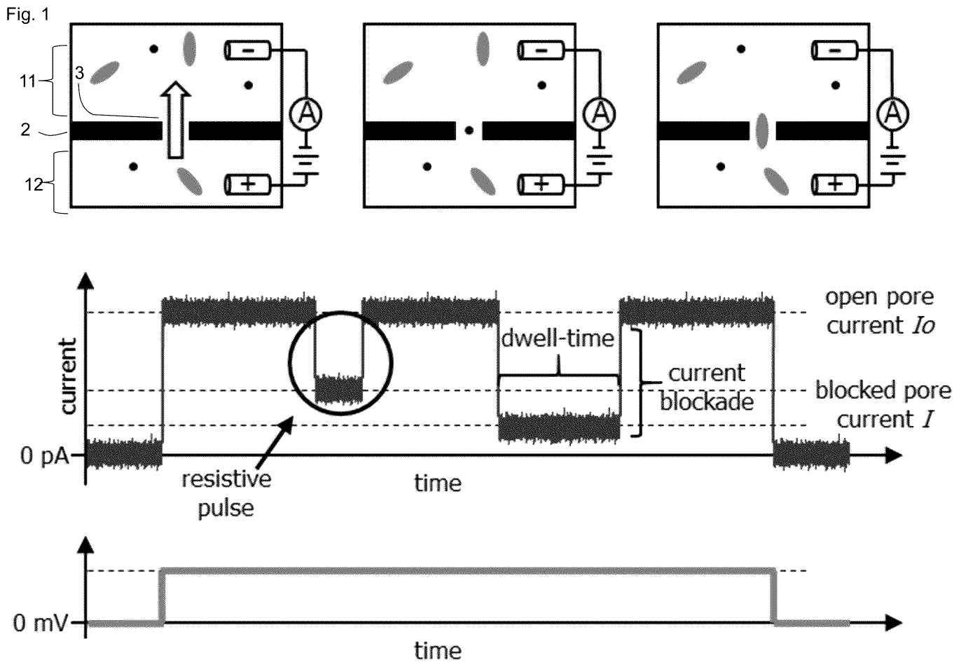

FIG. 1 shows a sketch of the principle of single-molecule sensing through nanopores. A constant potential difference ΔE across an insulator drives an ionic current through the pore. A single analyte molecule in the pore partially blocks the current (resistive pulse). Both the depth of the blockage, or residual current, and the duration and temporal variations of this current signal carry information about the analyte.

In both cases (biological and non-biological nanopores), the reduction in conductivity is measured as a change in ionic current caused by a constant voltage across the insulator in which the pore is the sole (or dominant) electrically conducting junction. These signals, called resistive pulses, correspond to individual analyte molecules entering the pore and interacting with the inner wall of the pore—and possibly, but not necessarily, translocating through the pore from one side of the insulator to the other.

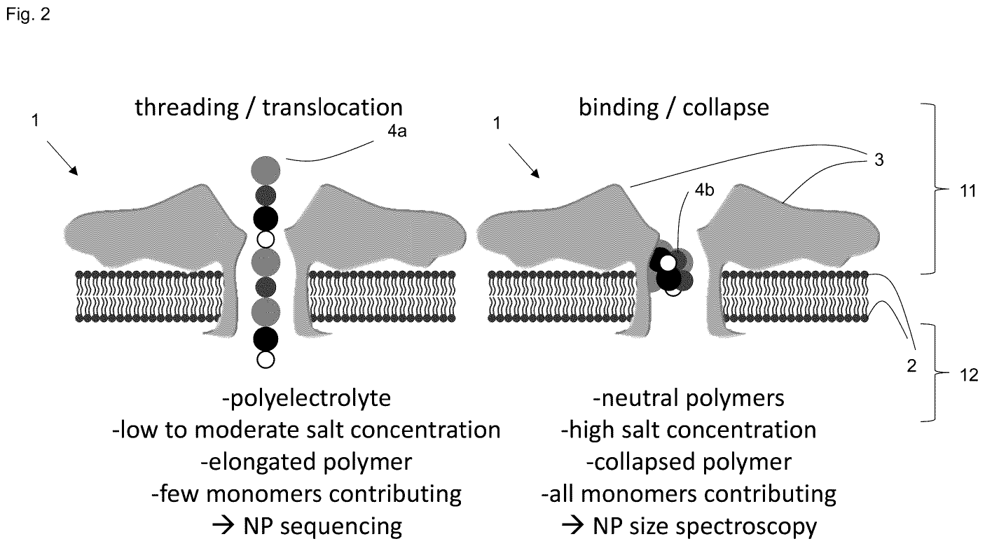

If the analyte is a polymer (e.g., a peptide, polynucleotide, or synthetic polymer such as poly(ethylene glycol)), two regimes must be distinguished, as shown in FIG. 2: in the threading regime, the polymer is stretched and few of its monomers contribute to the resistance change. In this regime, the current signal is sensitive to the identity of the monomers in the narrowest part of the pore and can therefore be used for sequencing if the polymer is threaded through the pore in a regular manner, i.e., at as uniform a rate as possible. In the collapsed regime, on the other hand, all monomers are present in the pore at the same time, so that the current decay is approximately proportional to molecular volume, although other, more subtle factors may also be involved. The collapsed regime has been used for nanopore-mediated determination of the molecular size distribution of neutral synthetic polymers (Baaken et al. 2015). It is assumed that non-specific binding of the collapsed polymer to the pore wall occurs in this regime (Binding regime;

Talarimoghari, M., G. Baaken, R. Hanselmann, and J. C. Behrends. 2018. size-dependent interaction of a 3-arm star poly(ethylene glycol) with two biological nanopores. Eur. Phys. J. E. 41:6288-8. doi:10.1140/epje/i2018-11687-6). FIG. 2 shows the two regimes of polymer-nanopore interaction. The threading/translocation regime is favored when long polyelectrolyte chains relative to the pore length interact with the pore at low to moderate salt concentrations (0.1 to 0.3 M KCl), employing relatively high electric voltages (>50 to >100 mV) to move the polymer through the pore in the electric field. The collapsed/bonded regime (also: trapping regime, since here the pore acts as a molecular trap) typically occurs under conditions of high salt concentration (e.g., 4 M KCl), does not require a compelling intrinsic charge of the analyte, and tends to require lower voltages (up to 50 mV) for charged analytes such as proteins, peptides, and polynucleotides, while higher voltages favor the translocation regime. The collapsed/bonded regime can only be used for polymers that are short enough or and/or sufficiently collapsed to fully occupy space in the pore. Binding and trapping of a polymer in the pore is also possible for charged polymers and also for polymers in the uncollapsed or not fully collapsed state, provided they are not too long for the pore. From the studies underlying the present invention, it was found that performing the current measurement method (step b) in claim 1) in the collapsed regime (also: collapsed, binding or trapping regime) is particularly advantageous.

While DNA sequencing by biological nanopores in the translocation/threading regime is well established and commercially available (see https://nanoporetech.com), peptide recognition and differentiation using nanopores is a nascent technique, with protein sequencing using nanopores a long-term goal that has yet to be achieved.

Peptides were threaded through biological protein nanopores such as the bacterial toxins aerolysin and alpha-hemolysin relatively early, but the interaction times were too short and the signal-to-noise ratio too low to distinguish between different peptides, let alone obtain sequence information. In the meantime, biological nanopores have been used to detect and differentiate peptides and proteins even in their native or folded state. Known is the ability of Frageatoxin (FraC) pores to distinguish between two forms of endothelin that differ only in two amino acid positions. (Huang, G., A. Voet, and G. Maglia. 2019. FraC nanopores with adjustable diameter identify the mass of opposite-charge peptides with 44 dalton resolution. Nat Comms. 10:347-10. doi:10.1038/s41467-019-08761-6.)

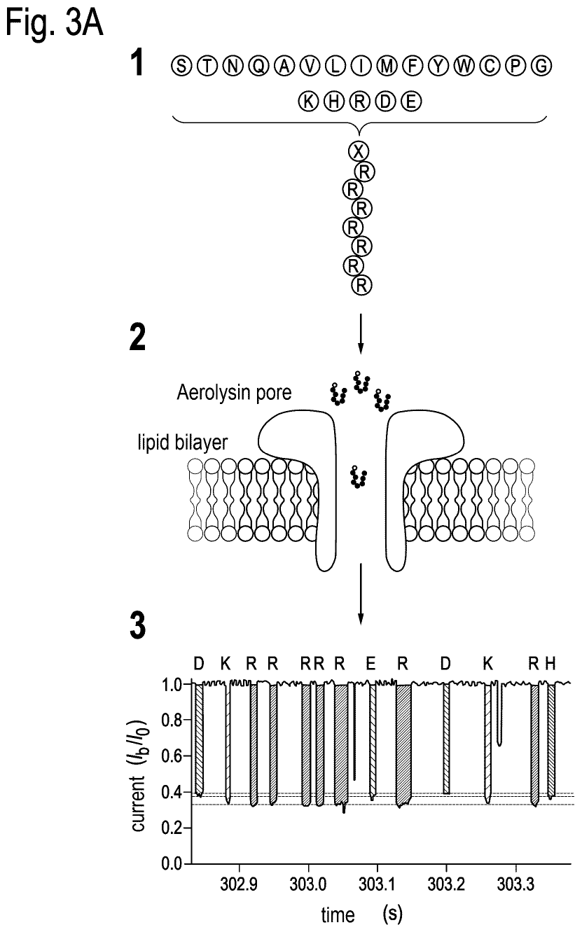

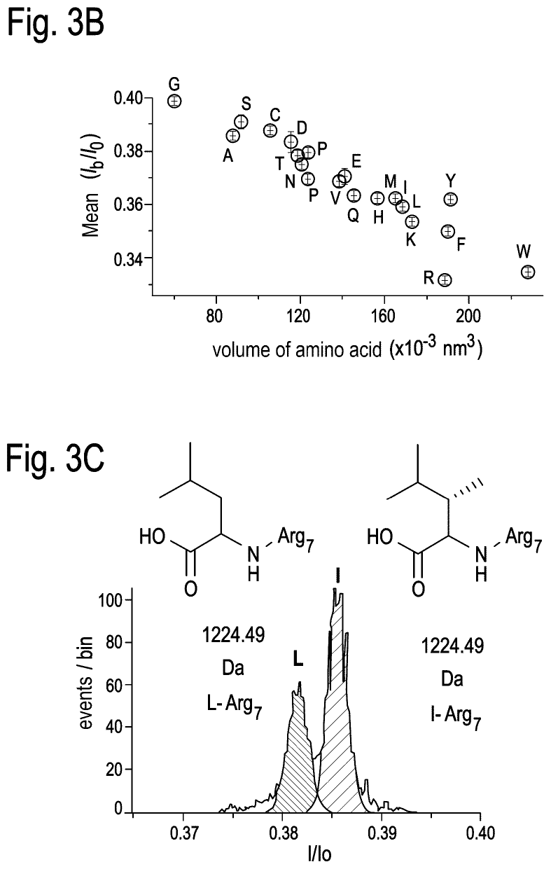

The well-documented superiority of the sensitivity of the aerolysin pore in the trapping/collapse regime, originally shown for poly(ethylene glycol) (Baaken et al. 2015), led to renewed interest in using this pore for peptide sizing. It was shown that the length of homoarginine peptides can be readily determined with this pore with an accuracy of one amino acid (Piguet et al. 2018). Furthermore, it was determined that the substitution of a single terminal residue in an octa-arginine peptide by one of the 20 proteinogenic amino acids can be detected and thereby differentiated between them, with sufficiently good discrimination of peptides even of the same mass (see FIG. 3, Ouldali et al. 2020). FIG. 3 shows the recognition of the twenty proteinogenic amino acids using the aerolysin nanopore. A: 1: peptide design 2: peptide-pore interaction. Current trace in the presence of a mixture of R₇+D,K,R,E,H. B: plot of relative current vs. volume of amino acid. C: >95% discrimination between the structural isomers R₇ −L and R₇ −I by high resolution measurement on the MECA platform (Ouldali et al. 2020).

The references cited here are: Baaken et al, 2015 “High-Resolution Size-Discrimination of Single Nonionic Synthetic Polymers with a Highly Charged Biological Nanopore”, ACS nano, VOL. 9, NO. 6, 6443-6449. Piguet et al., 2018, “Identification of single amino acid differences in uniformly charged homopolymeric peptides with aerolysin nanopore,” Nature Communications; 9, 966. Ouldali et al, 2020, “Electrical recognition of the twenty proteinogenic amino acids using an aerolysin nanopore,” Nature Biotechnology, VOL 38, 176-181.

In US 2019/0317006 A1, it was proposed to distinguish different peptides of a mixture from each other by nanopore size spectroscopy and using an aerolysin nanopore.

It is the object of the present invention to provide a technical solution for the identification of a sequence of monomer building blocks of a biological or synthetic heteropolymer, in particular a peptide or protein.

This object is solved according to the invention by the method according to claim 1, the use of a nanopore according to claim 12, the computer-implemented method according to claim 13, the program code stored on a data carrier according to claim 14, and the data processing system according to claim 15. Preferred embodiments of the invention are objects of the subclaims.
The method according to the invention is used to identify a sequence of monomer building blocks of a biological or synthetic heteropolymer, and comprises the following steps:
   (a) carry out a fragmentation method in which the heteropolymer is fragmented, in particular enzymatically, chemically and/or physically, thereby obtaining a fragment mixture whose fragments are molecules having different sequence segments of the heteropolymer;
   (b) perform a current measurement method in which current signals of a current through the channel of a single nanopore, or a current passing in parallel through a plurality or plurality of channels of a plurality or plurality of nanopores, are detected, each current signal being based on the interaction of a fragment with the channel of the nanopore, the current signals being characteristic of the different fragments, wherein a representative set of characteristic current signals representing the fragment mixture is determinable;
   (c) perform an evaluation method in which a sequence of monomer building blocks of the heteropolymer is determined from the representative set of characteristic current signals.

In a preferred embodiment of the method according to the invention, the fragments of the fragment mixture are obtained by successive degradation of the heteropolymer. Preferably, the successive degradation of the heteropolymer provides that the heteropolymer is chain-shaped and has positions 1 (chain start) to n (chain end) of the chain, and that the chain, starting from one end, is shortened stepwise by one monomer building block to obtain length fragments, in particular essentially all length fragments n−(n−i) (here, i is a counter which is iteratively counted through according to i=i+1 according to i=1, 2, 3 . . . n−2, n−1, n, so that the length fragments have a total length of n−(n−1), n−(n−2) . . . to n−(n−n) monomer units), of a heteropolymer consisting of n monomer units, each length fragment having the sequence of monomer units identical to the heteropolymer starting from position 1 (chain start) to position n−(n−i). Such a fragment mixture is also referred to here as a “ladder” or a heteropolymer ladder, i.e. a “peptide ladder” if the heteropolymer is/features a peptide.

In this context, the monomer building blocks may belong to a set m of possible monomer building block species, e.g., in the case of eukaryotic proteins, a number n of amino acids (monomer building blocks) may form the protein (heteropolymer) (or a sequence thereof), which may be limited to the set m=21 of human proteinogenic amino acids (i.e., monomer building block species).

Instead of successive degradation, another degradation method can be used that yields the above-mentioned length fragments of the heteropolymer.

The sequence of monomer building blocks of the heteropolymer determined in step c) may be a part of the total sequence (partial sequence) of monomer building blocks of the heteropolymer, or, preferably, may be the total sequence of monomer building blocks of the heteropolymer.

Preferably, the heteropolymer is a peptide. Preferably, the fragmentation method is an Edman degradation or includes an Edman degradation. Further, the fragmentation method may be designed to provide for cleavage of the protein by endopeptidases to peptides, and in particular treatment of the peptides by exopeptidases to obtain the peptide ladder. Preferably, the method according to the invention comprises the following steps:
in particular in each case preferably in step b):
   determine residual current values (of the current signals) from the measured data, where a residual current describes the interaction of one of the different fragments of the heteropolymer with a nanopore;
   statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence—preferably unambiguously, but in any case sufficiently for a desired structure elucidation or structure prediction;
in particular in each case preferably in step c):
   sort the characteristic residual current values by their magnitude into a residual current value sequence and determining the current value differences of successive current values of the residual current value sequence; and
   assign the current value differences to monomer building block types of the heteropolymer on the basis of previously known correlation data containing information about which monomer building block type is represented by which current value amount in order to carry out the determination of the sequence of monomer building block types (=determination of the sequence of monomer building blocks of the heteropolymer).

A characteristic residual current value denotes the measurement results of the current value measurement, which results from the interaction of a certain fragment, which is characterized by the characteristic residual current value, with the nanopore. In particular, the characteristic residual current value includes the residual current value amount attributable to the corresponding current signal. The characteristic residual current value may also be a vector-valued quantity which, in addition to the residual current value amount, includes other components whose number determines the dimension of the vector-valued quantity. Such components can be a time duration of the current signal or another quantity describing the time course of this current signal, or can be parameters describing an interpolation curve which is used to describe the current signal.

A characteristic residual current value describes in each case one fragment type, in particular fragment size, of the number n of fragment types of a fragment mixture formed from the heteropolymer. Example: a fragment mixture formed as a peptide ladder contains a total of n fragment types, starting from a peptide with n amino acids as monomer building blocks. The peptide solution containing the fragment mixture usually contains a large number of fragments of each fragment type (peptide type). Ideally, a fragment mixture obtained by 100% efficient fragmentation of one of a starting set having a total number M of the peptide to be sequenced also contains a number M of fragments of each of the n fragment types of the peptide. When “fragment” is referred to in this application, depending on the context, it may mean in particular the fragment type.

A “representative set of characteristic residual current values”, which can be derived in particular from the total number of measured residual current values, describes a plurality or multiplicity, preferably the totality, of the characteristic residual current values determined for the fragment mixture by means of the current value method mentioned in step b).

Preferably, the method according to the invention is defined as an extended method serving to determine a sequence of a protein, comprising the steps of.
i) cleavage of the protein, in particular by enzymatic and/or chemical and/or physical cleavage, to obtain peptides as cleavage products of the protein; optionally: recovery of the peptides by chromatographic or electrophoretic separation of a peptide mixture obtained by the cleavage;
ii) Application of the method according to the invention for determining the sequence of amino acids (monomer building blocks) of at least one, in particular each, of the peptides (heteropolymer);
(iii) performing a recognition method for recognizing the sequence of the protein, wherein the sequence of the protein is determined from the sequence of amino acids of the at least one peptide.

The method according to the invention or the above-mentioned embodiment of the method according to the invention can advantageously be used to elucidate the, in particular complete, primary structure of a macromolecule, in particular a biological macromolecule, in particular a protein, wherein the biological macromolecule comprises various heteropolymers, in particular is formed from various heteropolymers bonded to one another:

Preferably, the method according to the invention is defined as an extended method used to determine the primary structure of a macromolecule, in particular a protein, comprising the steps of.
i) cleavage of the macromolecule, in particular protein, in particular by enzymatic and/or chemical and/or physical cleavage, to obtain heteropolymers, in particular peptides, as cleavage products of the macromolecule; optionally: obtaining heteropolymers, in particular the peptides, by separation, in particular chromatographic or electrophoretic separation, of a heteropolymer mixture, in particular peptide mixture, obtained by the cleavage;
ii) Application of the method according to the invention for determining a sequence of monomer building blocks, in particular amino acids, of at least one, in particular each, of the heteropolymers, in particular peptides;
iii) perform a macromolecule recognition method, in particular protein recognition method, in which the primary structure of the macromolecule, in particular protein, is determined from the sequence of the at least one heteropolymer, in particular peptide, wherein the macromolecule is preferably the DNA, RNA, protein, peptide or any synthetic polymer.

The method according to the invention can be designed to determine the complete sequence of the monomer building blocks from which the heteropolymer or the macromolecule is built, or one or more partial sequences thereof.

The method according to the invention can be configured to determine a part of the complete sequence of monomer building blocks of which the heteropolymer is composed. If only part of the complete sequence of monomer building blocks of a heteropolymer is determined, the method according to the invention can in particular be used to implement a determination method in which the partial sequence of monomer building blocks of a heteropolymer determined by the method according to the invention is used to determine which previously known heteropolymer has been determined from a set T (1 to T) of previously known different heteropolymers (namely different with respect to their sequence). “Pre-known” means here that the nearly complete, or complete sequence of monomer building blocks of each pre-known heteropolymer is known. The partial sequence determined by the method according to the invention represents a “fingerprint” of the heteropolymer to be determined from the previously known set of heteropolymers, i.e. a feature which makes the heteropolymer sought uniquely identifiable with respect to the other heteropolymers of sets 1 to T. The steps of such a determination method can be described as follows:
i) Providing the information about the pre-known sequence of each heteropolymer of a set of 1 to T different heteropolymers;
ii) Taking a heteropolymer to be determined which is identical with exactly one heteropolymer of this set of 1 to T different heteropolymers, wherein in particular it is not known with which heteropolymer of this set the heteropolymer to be determined is identical;
iii) Performing the method according to the invention to determine a partial sequence of the heteropolymer to be determined;
iv) comparing the partial sequence determined in iii) with the previously known sequences of all heteropolymers of the set of 1 to T different heteropolymers and determining the heteropolymer sought from the set of previously known heteropolymers on the basis of the partial sequence which makes the heteropolymer sought uniquely identifiable with respect to the other heteropolymers of the set of 1 to T.

The said determination method allows the complete sequence of a sought heteropolymer to be determined without having to elucidate the complete sequence of the sought heteropolymer by means of the method according to the invention, if the sought heteropolymer originates from a set T of previously known heteropolymers each having a previously known sequence, a partial sequence—in the manner of a fingerprint—uniquely identifying the sought heteropolymer with respect to the remaining heteropolymers of this set. In this scenario, the determination method is the more efficient way to determine the complete sequence of the sought heteropolymer, compared to the alternative of elucidating the complete sequence of the sought heteropolymer by means of the method according to the invention instead of the partial sequence of the sought heteropolymer.

Preferably, the nanopore is a biological nanopore, i.e., a pore-forming toxin or a porin.

Preferably, the nanopore is a solid-state nanopore or a hybrid of solid-state and biological and/or chemical components. A solid, in particular a substrate, may include or be formed from at least one of the following materials: SiNx, SiO₂, HfO₂, MoS₂, CNT, graphene, nanopipettes. Biological or chemical components may, each preferably, include or consist of at least one of the following: Pore-forming toxins, porins, βeta-barrel proteins, alpha-helical membrane proteins, DNA origami structures. Hybrids, combinations of all of the above components are possible.

Preferably, the fragmentation of the heteropolymer is carried out by enzymes. Preferably, these are endo/exo peptidases for proteins/peptides and common restriction enzymes (nucleases) for DNA. The person skilled in the art will choose an enzyme set up for this purpose depending on which sequence he wants to cut.

Possible peptidases are mentioned, for example, in: https://www.ebi.ac.uk/merops/Possible nucleases are mentioned, for example, in: https://wikivisually.com/wiki/List_of_restriction_enzyme_cutting_sites %3A_Bst % E2%80% 93Bv#Whole_list_navigation

Preferably, fragmentation of the heteropolymer is done chemically and non-enzymatically. For proteins/peptides, the Schlack-Kumpf and Edman degradation can be used. For DNA, enzymes are usually used.

Preferably, the fragmentation of the heteropolymer takes place by physical means, e.g. by exposure to heat, cold, sound waves, electromagnetic radiation, in particular infrared, ultraviolet or X-ray radiation, microwaves or visible light. Examples are documented in https://doi.org/10.1073/pnas.0901422106 or https://doi.org/10.1007/s13361-017-1794-9 and https://doi.org/10.1002/mas.20214.

Preferably, the nanopore is selected from the group of preferred nanopore proteins containing aerolysin, alpha-hemolysin, MspA, CsgG, VDAC or another protein from the family of beta-barrel proteins, as well as genetically optimized variants of these pore proteins.

The pore proteins and the other measurement conditions are thereby preferably optimized for an interaction of the analyte (the fragment) with the pore, which results in an interaction between analyte and pore that is optimally long-lasting for the respective analyte. A preferred embodiment of the nanopore is as follows: the nanopore is preferably an aerolysin pore, in particular a variant of the aerolysin pore. For this purpose, for example, the single molecule trap of the aerolysin pore can be adapted and optimized to the analyte by single point mutation in the dimension and depth of the potential well. In particular, this is done by the aerolysin variants R220S/A/C/K/H/E/D/Q/N, R288S/A/C/K/H/E/D/Q/N, R282S/A/C/K/H/E/D/Q/N, D222S/A/C/F/R/K/H/E/Q/N, D216S/A/C/F/R/K/H/E/Q/N, D209S/A/C/F/R/K/H/E/Q/N, K238S/A/C/F/R/D/H/E/Q/N, K242S/A/C/F/R/D/H/E/Q/N, K244S/A/C/F/R/D/H/E/Q/N, K246S/A/C/F/R/D/H/E/Q/N, E237S/A/C/F/R/D/H/K/Q/N E258S/A/C/F/R/D/H/K/Q/N E254S/A/C/F/R/D/H/K/Q/N, E252S/A/C/F/R/D/H/K/Q/N and any combinations thereof.

The aerolysin pore in its natural form (wild type) or as a variant thereof is particularly preferred for use as a nanopore in the context of the invention. The variant may be designed to differentiate and characterize fragments of heteropolymers that differ, for example, only by positional isomerism. Using the R220S variant of the aerolysin pore, for example, differentiation of positional isomerism derived from acetylation has been performed (“Resolving isomeric posttranslational modifications using a nanopore,” Tobias Ensslen, Kumar Sarthak, Aleksei Aksimentiev, Jan C. Behrends, bioRxiv 2021.11.28.470241; doi: https://doi.org/10.1101/2021.11.28.470241).

Translocation or passage of the analyte through the pore is not necessary, although it is permitted in principle. Rather, it is particularly advantageous if the same analyte visits its binding site in the pore for as long as possible, or revisits it several times and binds there after having left the molecular trap again in the direction of the entrance opening in the meantime. Preferably, therefore, “interaction” of the fragment (analyte, molecule) with the channel of the nanopore means that the fragment enters the channel but does not pass through the channel, which ultimately results in a non-destructive multiple determination of the same molecule.

By trapping the same analyte in the pore for as long as possible or repeatedly, a particularly precise determination of the characteristic residual current values by means of temporal signal averaging and a representative determination of the parameters of the time course of the current signal (variance, noise analysis) is made possible. It is understood that an interaction of analyte and pore should not last indefinitely, otherwise the accessibility of the pore for analyte molecules is reduced. This results in an optimal interaction duration adapted to the analyte, which can be achieved in particular by variant formation of the nanopore, preferably of the aerolysin.

From the investigations underlying the present invention, it was found that carrying out the current measurement method (step b) in claim 1) in the collapse regime (also: collapsed, binding or trapping regime) is particularly advantageous. The current measurement method carried out in step b) is preferably performed such that the fragment mixture is present in an electrolyte solution comprising, in particular, dissolved salts of the form AX, A₂ X and AX₂ etc., where substance A (e.g. selected from the alkali and alkaline earth metals Na, K, Cs, Rb, Li) provides the cation and substance X (e.g. selected from the halogens F, Cl, Br) provides the anion. The substance groups A and X may comprise further constituents in the sense of inorganic or organic derivatives of such salts (where, for example, substance A is a quaternary ammonium, imidazolium, phosphonium, pyridinium and pyrrolidinium ion such as e.g. tetramethylammonium, and substance X may be a nitrate, a sulfate, phosphate, an amino acid such as glutamate, a carboxylic acid such as gluconate, citrate, a (bi)carbonate, or a simple hydroxide). Preferably, the electrolyte solution may also comprise mixtures of different combinations of different salts.

The total salt concentration of the electrolyte solution in which the fragment mixture is present during the performance of the current measurement method is between 0.5 M and 20 M, preferably between 2 M and 10 M and particularly preferably between 3 M and 5 M. The fragment mixture can also be present in an ionic liquid as an alternative to an electrolyte solution. Such configurations of the electrolyte have the effect of optimally setting conditions such as charge shielding and solubility of the analyte in the electrolyte solution for the collapsed/bonded regime and the longest possible residence time of the analyte in the molecular trap of the pore, while at the same time achieving the highest possible signal-to-noise ratio of the current measurement.

The invention also relates to the use of a nanopore for carrying out the method of the invention for identifying a sequence of monomer building blocks of a biological or synthetic heteropolymer.

The invention also relates to a computer-implemented method for determining a sequence of monomer building blocks of a heteropolymer (heteropolymer sequence) from measurement data of a current measurement method containing information on current signals obtained upon interaction of different fragments formed from the heteropolymer with a nanopore, comprising the steps:
A) determine residual current values from the measured data, where a residual current describes the interaction of one of the different fragments of the heteropolymer with a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence unambiguously, but in any case sufficiently for a desired structure elucidation or structure prediction;
C) sort the characteristic residual current values by their magnitude into a residual current value sequence and determining the current value differences of successive current values of the residual current value sequence; and
D) assign the current value differences to monomer building block types of the heteropolymer based on pre-known correlation data containing information about which monomer building block type is represented by which current value amount to perform the determination of the sequence of monomer building block types (determination of the sequence of monomer building blocks of the heteropolymer).

The invention also relates to a computer program code which is stored on a data carrier and which determines a sequence of monomer building blocks of a heteropolymer (heteropolymer sequence) from the measurement data of a current measurement method when executed by the central processor of a computer, the measurement data containing information about current signals which are determined upon the interaction of different fragments formed from the heteropolymer with a nanopore, comprising the respective steps implemented by the program code:
A) determine residual current values (of the current signals) from the measured data, wherein a residual current describes the interaction of one of the different fragments of the heteropolymer with a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence unambiguously, but in any case sufficiently for a desired structure elucidation or structure prediction;
C) sort the characteristic residual current values by their magnitude into a residual current value sequence and determining the current value differences of successive current values of the residual current value sequence; and
D) assign the current value differences to monomer building block types of the heteropolymer based on pre-known correlation data containing information about which monomer building block type is represented by which current value amount to perform the determination of the sequence of monomer building block types (determination of the sequence of monomer building blocks of the heteropolymer).

The invention also relates to a data processing system for determining a sequence of monomer building blocks of a heteropolymer (heteropolymer sequence) from the measurement data of a current measurement method containing information on current signals determined upon interaction of different fragments formed from the heteropolymer with a nanopore, comprising a computer with a central processor, and a program code, in particular the program code according to the invention, wherein the computer is programmed to perform the following computer-implemented steps:
A) determine residual current values (current signals) from the measurement data, where a residual current describes the interaction of one of the different fragments of the heteropolymer with a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence unambiguously, but in any case sufficiently for a desired structure elucidation or structure prediction;
C) sort the characteristic residual current values by their magnitude into a residual current value sequence and determining the current value differences of successive current values of the residual current value sequence; and
D) assign the current value differences to monomer building block types of the heteropolymer based on pre-known correlation data containing information about which monomer building block type is represented by which current value amount to perform the determination of the sequence of monomer building block types (determination of the sequence of monomer building blocks of the heteropolymer).

The evaluation method, in which the sequence of the monomer building blocks of the heteropolymer is determined from the representative set of the characteristic current signals, preferably provides for the computer-implemented steps:
A) determine residual current values (current signals) from the measurement data, where a residual current describes the interaction of one of the different fragments of the heteropolymer with a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence preferably unambiguously, but in any case sufficiently for a desired structure elucidation or structure prediction;
C) sort the characteristic residual current values by their magnitude into a residual current value sequence and determining the current value differences of successive current values of the residual current value sequence; and
D) assign the current value differences to monomer building block types of the heteropolymer, preferably on the basis of previously known correlation data containing information about which monomer building block type is represented by which current value amount, in order to carry out the determination of the sequence of monomer building block types (determination of the sequence of monomer building blocks of the heteropolymer).

In steps A) to D), it is possible that the representative set of characteristic residual current values cannot unambiguously describe the heteropolymer because, for example, only part of the heteropolymer was fragmented or because not all characteristic residual current values could be unambiguously determined. In this case in particular, a prediction algorithm can be used to indicate from the incomplete data, in particular from an incomplete representative set of characteristic residual current values, a probability or an evaluation factor for evaluating the reliability of a primary structure of the heteropolymer determined by estimation. In this context, the prediction algorithm may have been determined by machine learning using, in particular, labeled training data. The labeled data may contain variations of incomplete representative sets of the characteristic residual current values of previously known heteropolymers. The prediction algorithm may include an artificial neural network, in particular a convolutional neural network (CNN), which may be trained by the labeled training data. The prediction algorithm may also implement unsupervised machine learning.

Further preferred embodiments of the objects according to the invention result from the following description of the embodiment examples in connection with the figures. Identical reference signs designate essentially identical components or method steps.

FIG. 1 shows a sketch of the principle of single molecule detection by nanopores shown, which can be used in the method 100 according to the invention.

FIG. 2 shows the two possible regimes of a polymer-nanopore interaction.

FIG. 3 shows the detection of the twenty proteinogenic amino acids (aa) using the aerolysin nanopore, in particular according to the prior art.

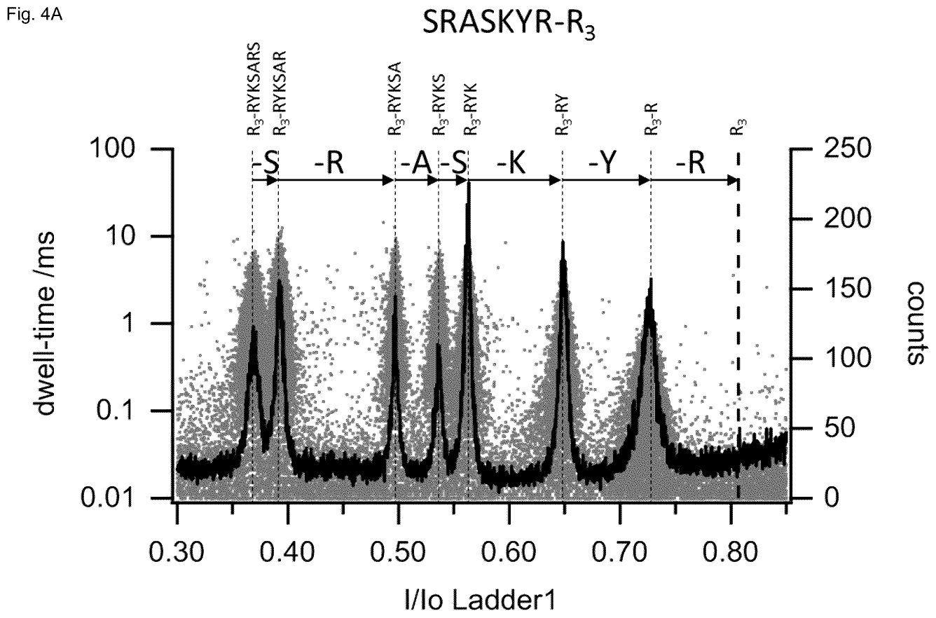

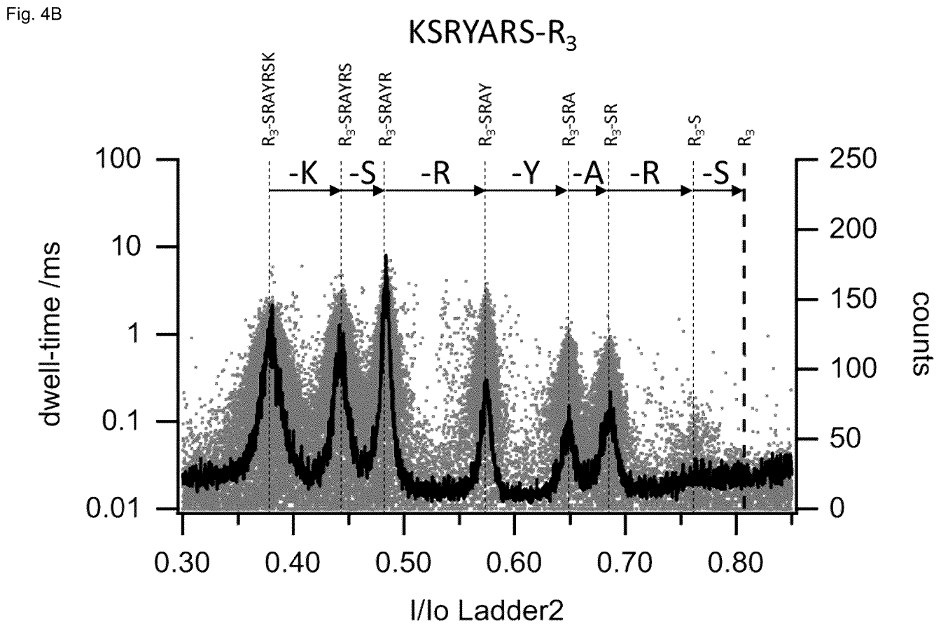

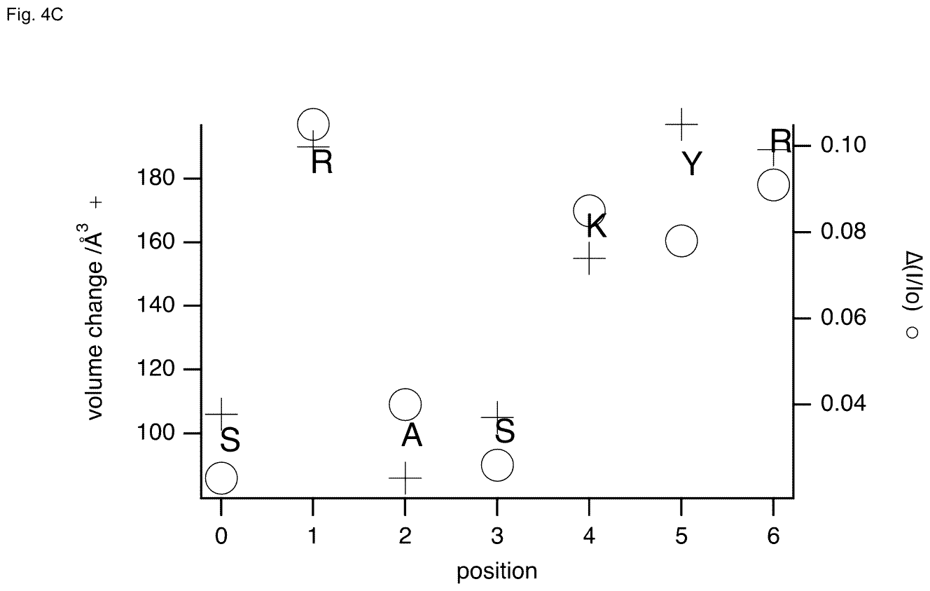

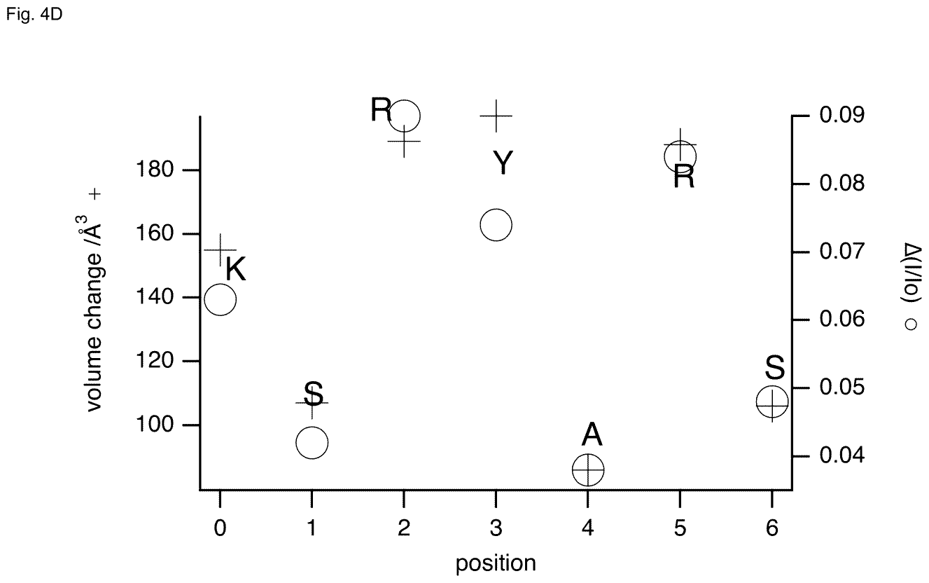

FIG. 4 shows measurement proofs for an exemplary process designed according to the invention.

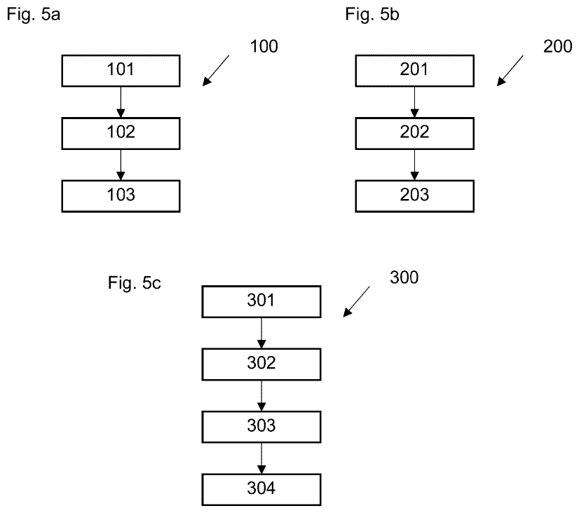

FIGS. 5a, 5b and 5c each show embodiments of the process according to the invention and of its components.

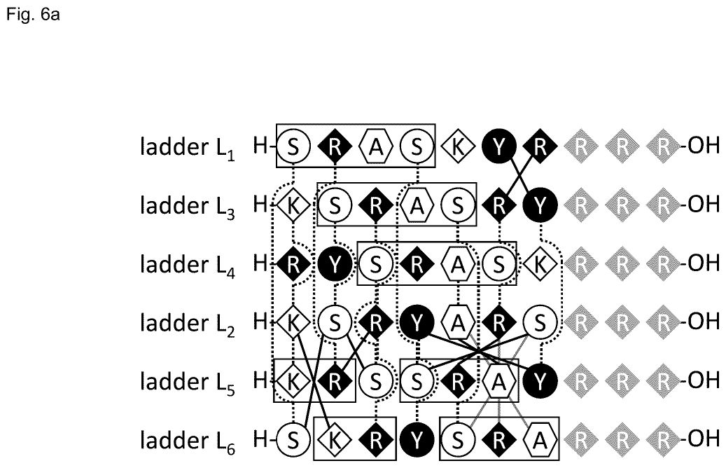

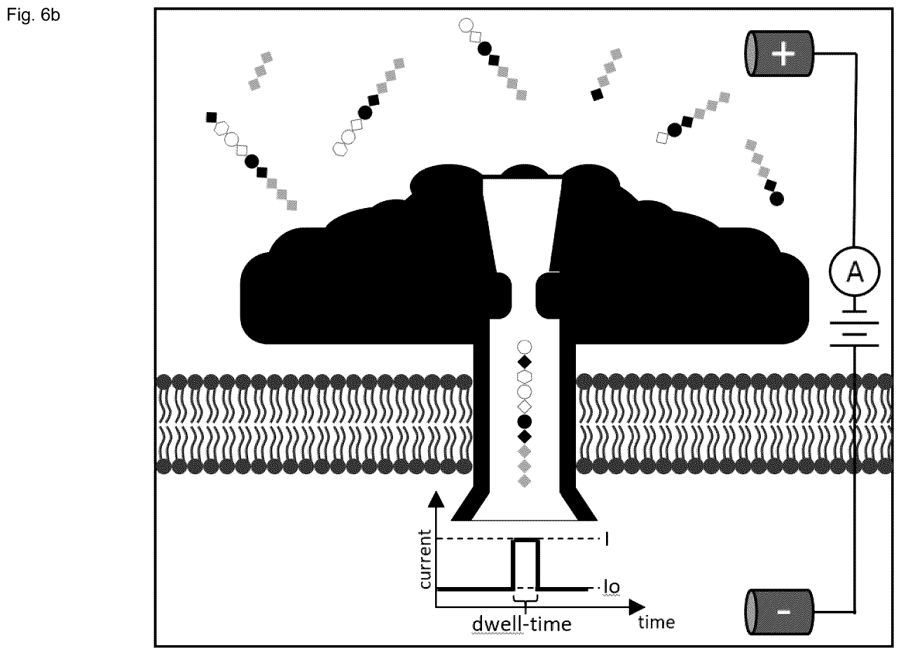

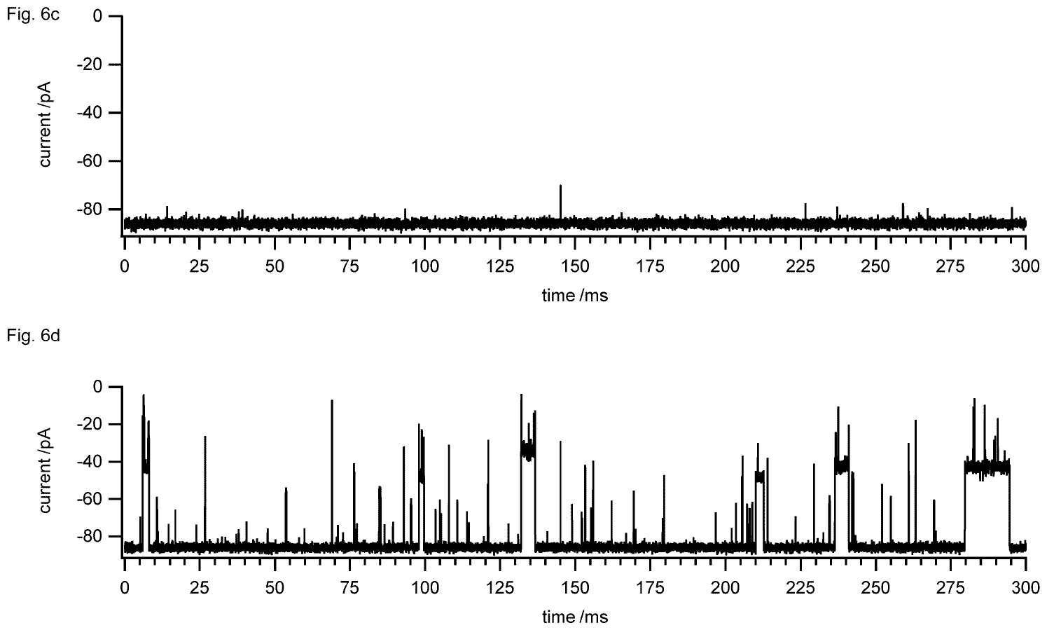

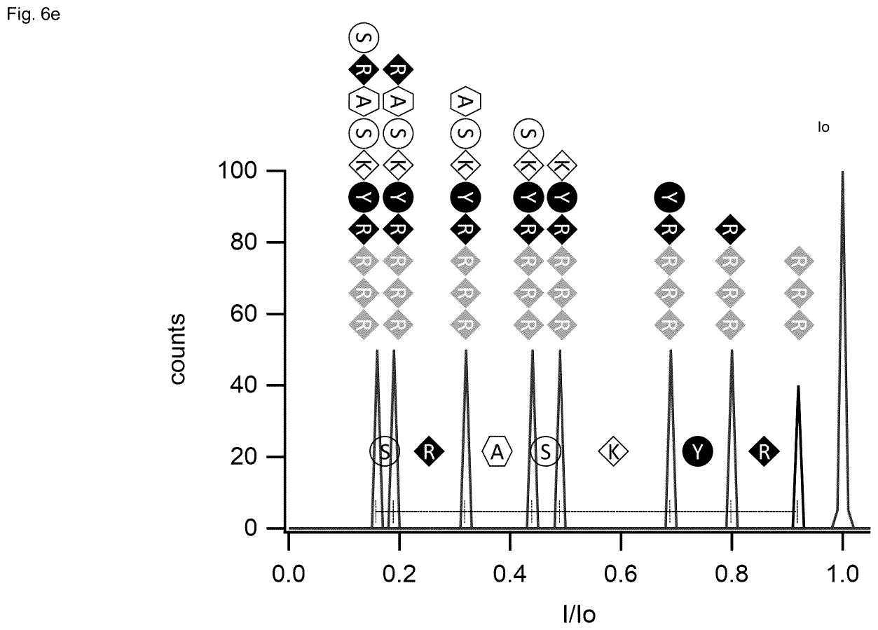

FIG. 6a shows, with reference to an embodiment of the invention: sequences of the six heterodeca peptides that constitute the ladder start peptide.

FIG. 6b shows, with reference to an embodiment of the invention: a schematic diagram of the experimental setup.

FIG. 6c shows, with reference to an embodiment of the invention: a control trace in 4 M KCl.

FIG. 6d shows, with reference to an embodiment of the invention: an exemplary measurement curve after addition of the peptide ladder L1 with all peptides in equimolar concentration.

FIG. 6e shows, referring to an embodiment of the invention: a schematic level histogram averaged over the main level for a peptide ladder sequencing experiment.

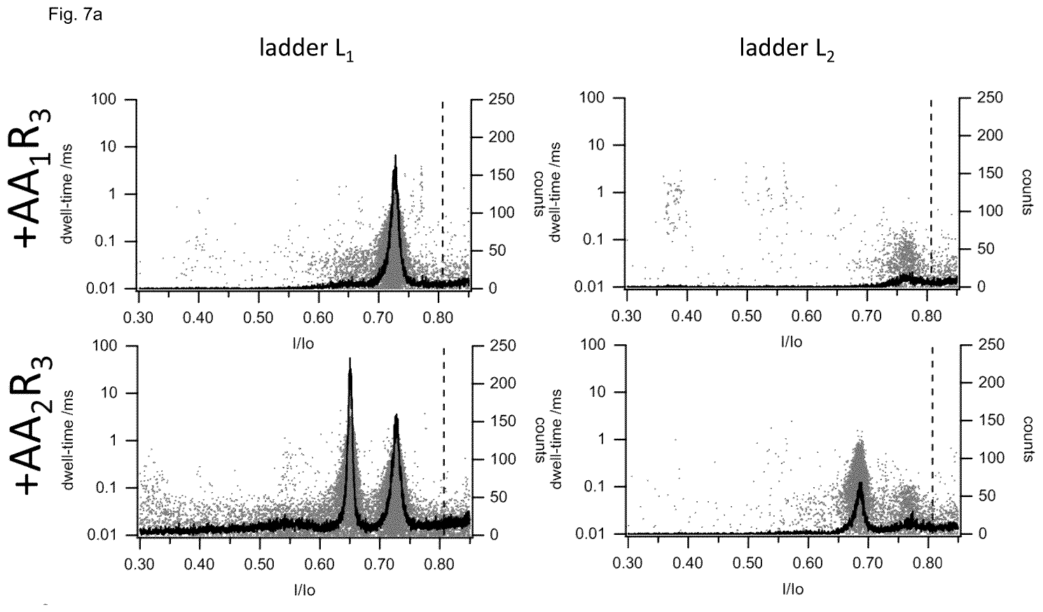

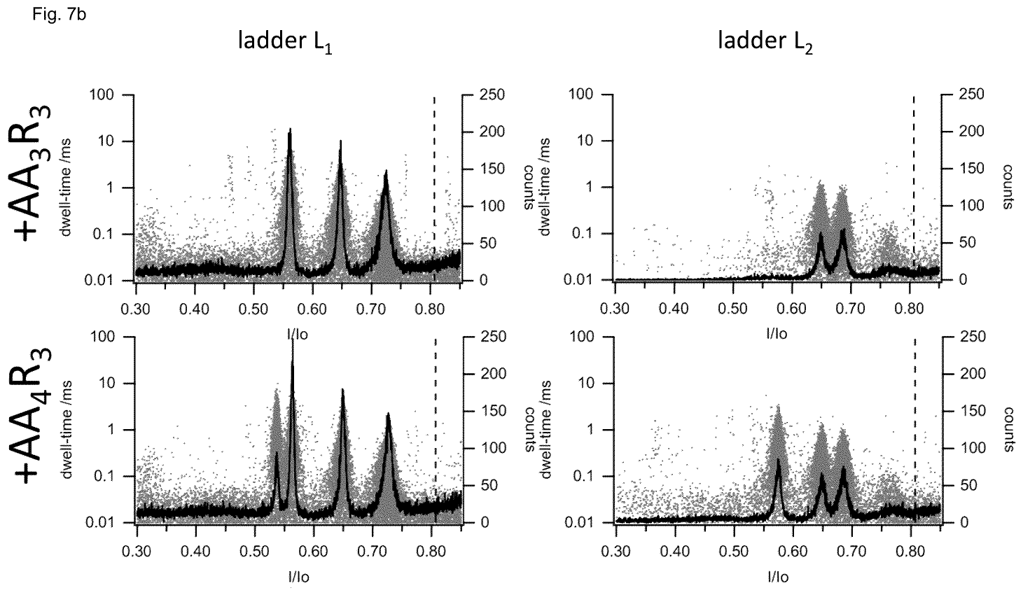

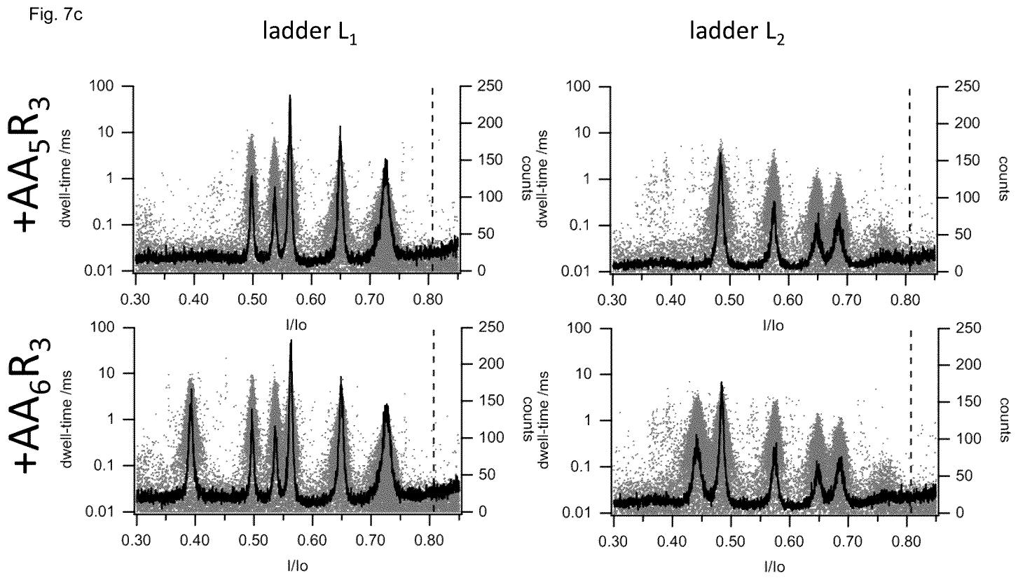

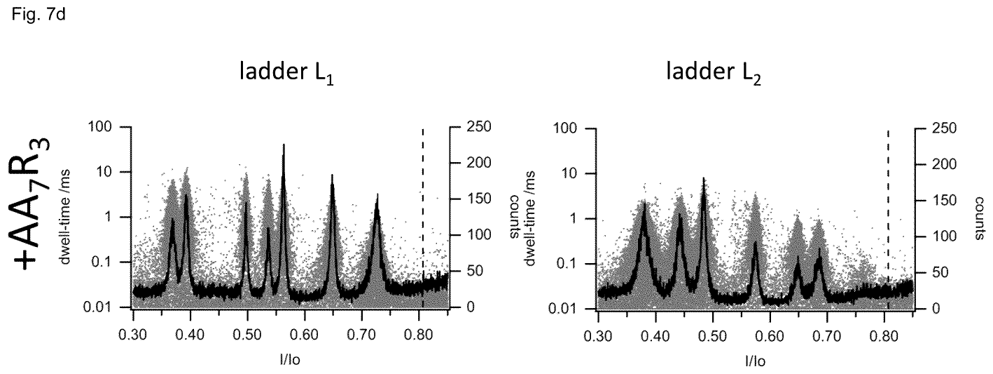

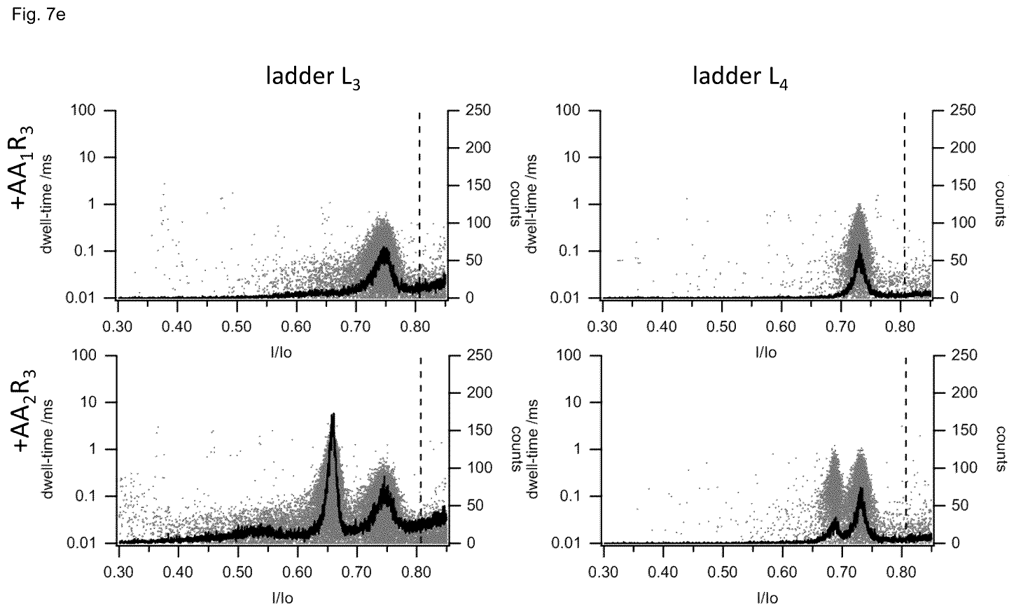

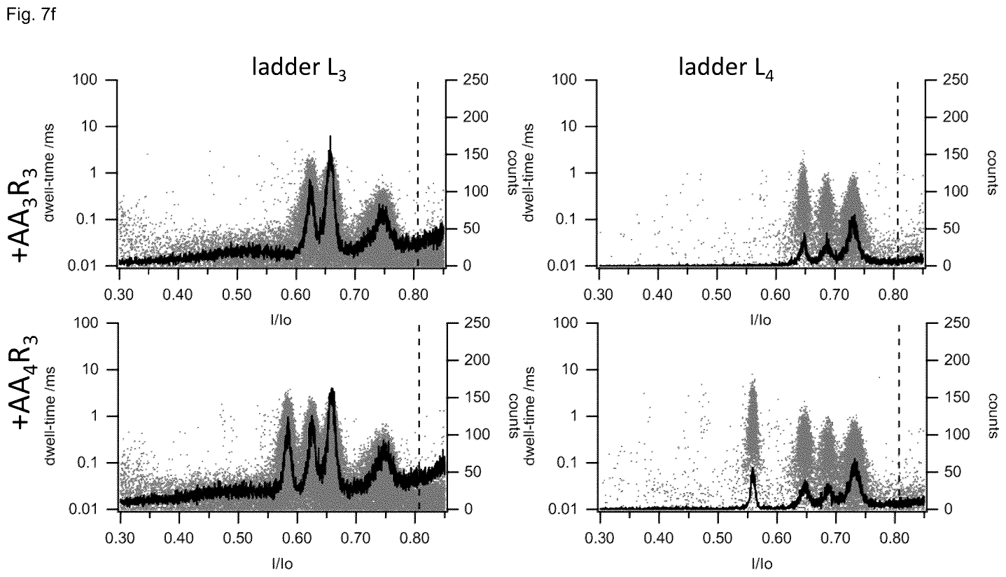

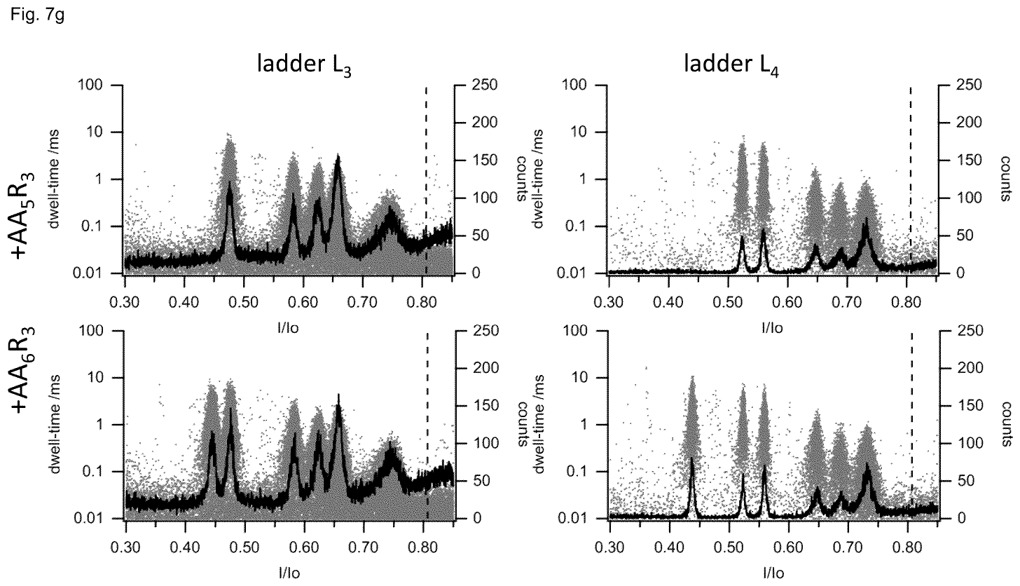

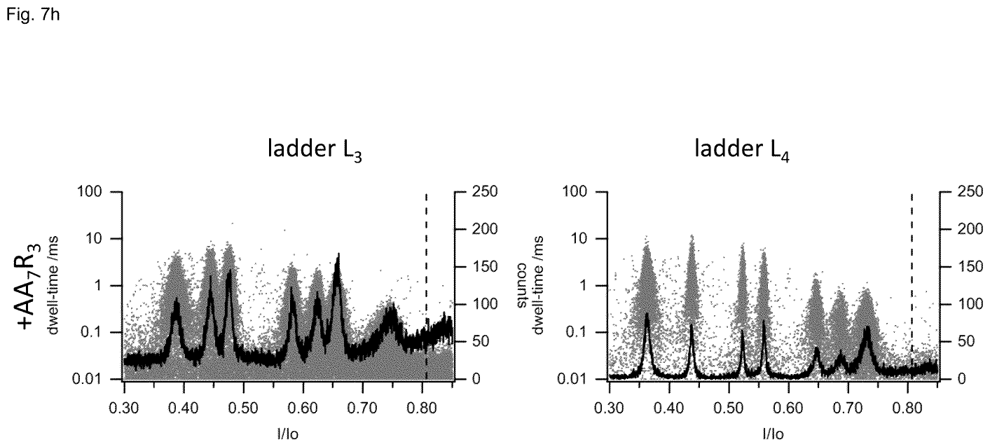

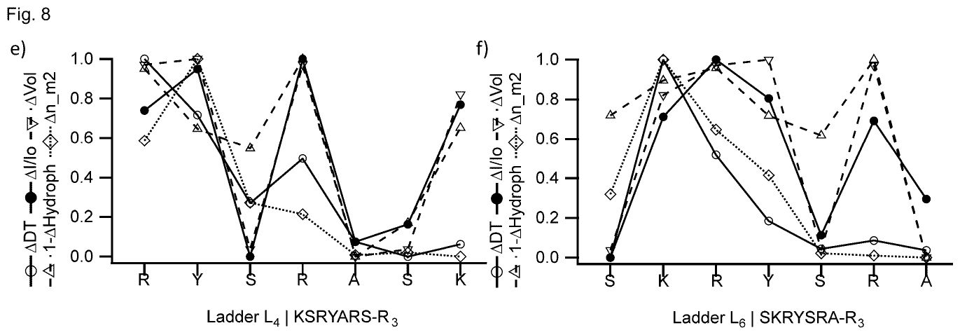

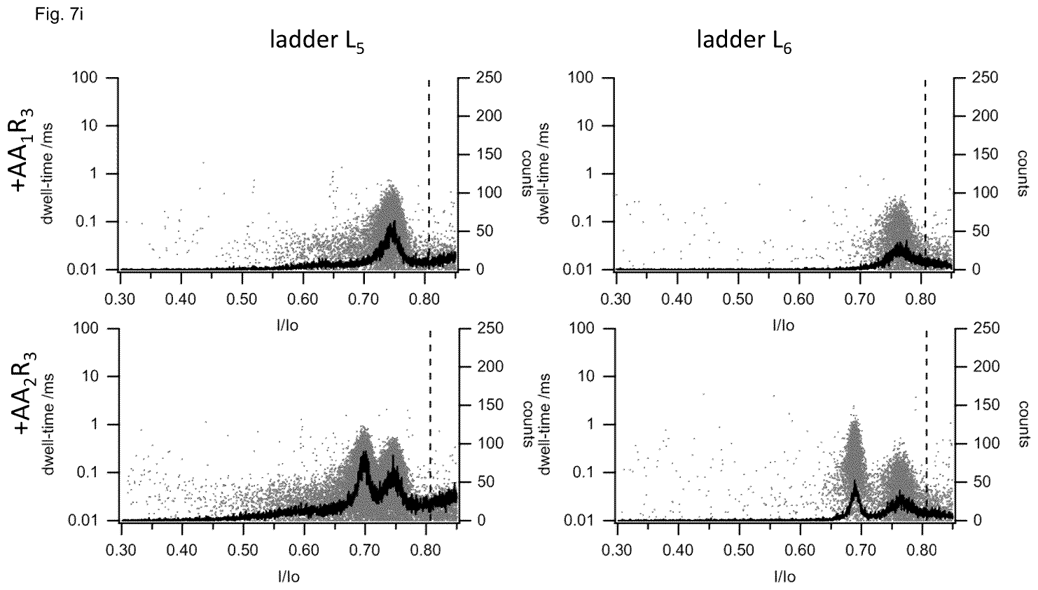

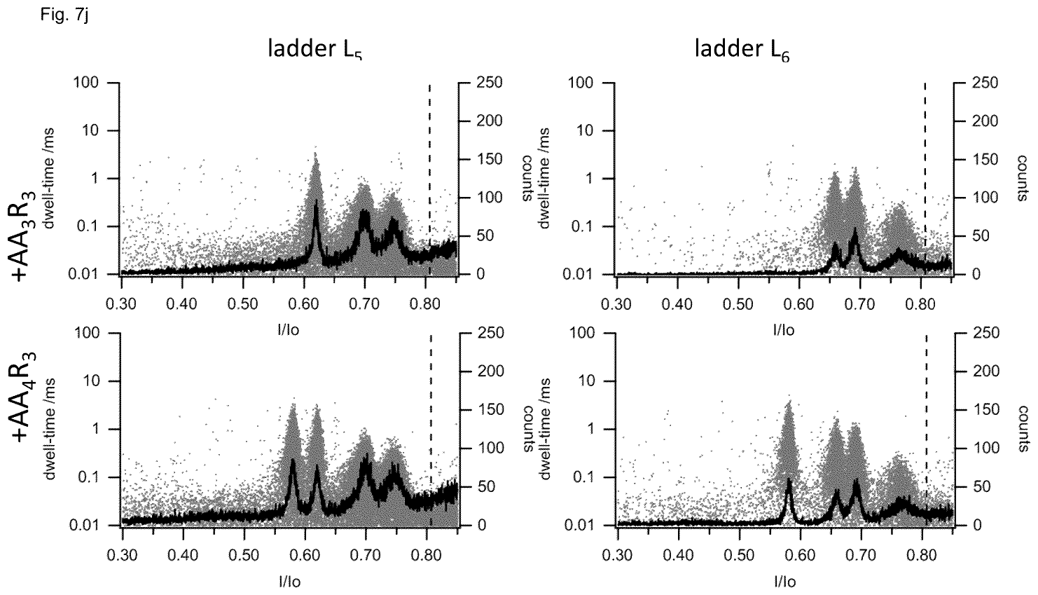

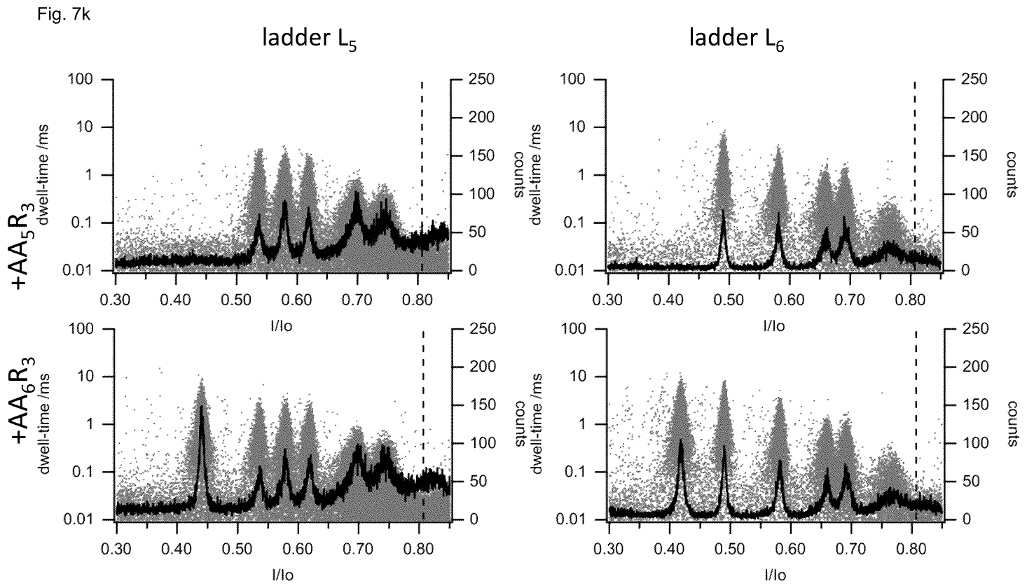

FIG. 7 shows, with reference to an embodiment of the invention: residence time scatter plots over the residual pore current I/Io (red) with superimposed level histograms averaged over the main level (black) for all six peptide conductors.

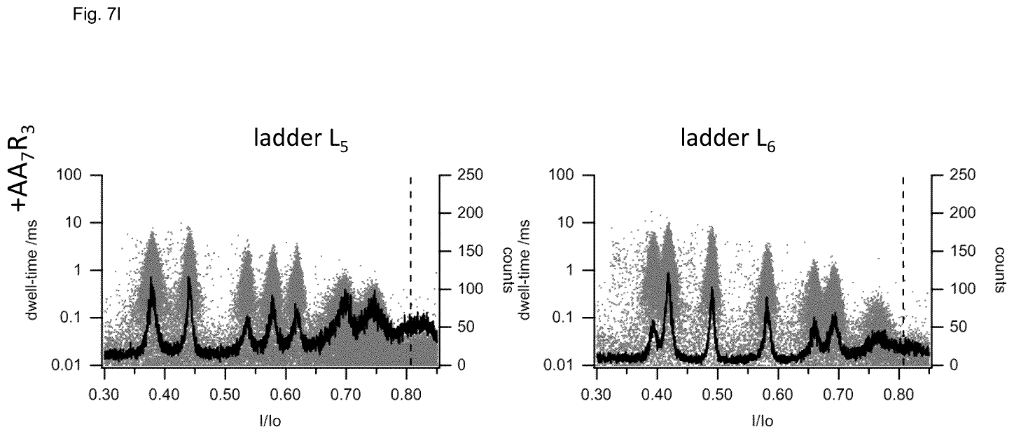

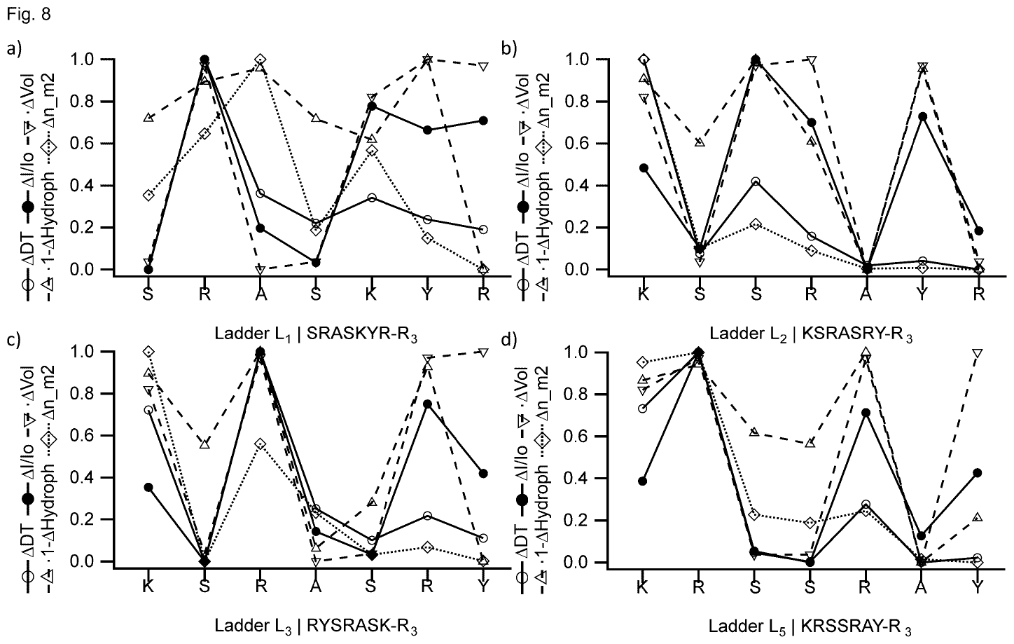

FIG. 8 shows, with reference to an embodiment of the invention: Data correlation plots for all six peptide ladders.

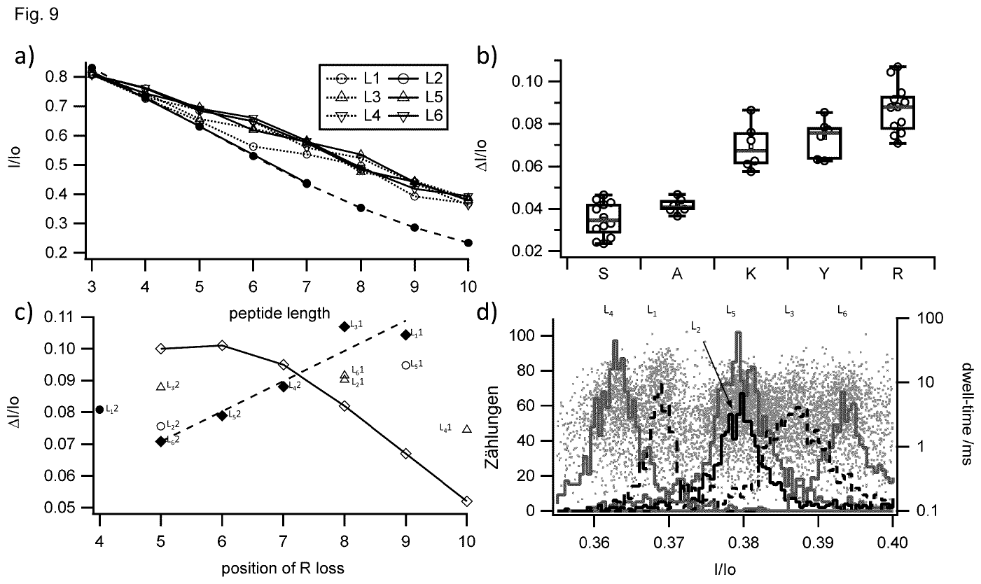

FIG. 9a shows, with respect to an embodiment of the invention: reproducibility of I/Io of homo-arginine peptides R3, R4, R5, R7 (blue) compared to R3-R7 of Piguet et al. 2018 (red), and ladders L1 (green, solid line, circle), L3 (green, dashed, pointing triangle), L4 (green, dotted, pointing triangle), L2 (pink, solid line, circle), L5 (pink, dashed, pointing triangle), L6 (pink, dotted, pointing triangle).

FIG. 9b shows, with reference to an embodiment of the invention: ΔI/Io boxplot for each cleaved amino acid type with median (blue) and mean (white).

FIG. 9c shows, with reference to an embodiment of the invention: ΔI/Io values for arginine cleavage classified by nearest neighbor aa of arginine as C-terminal aa (alanine blue, arginine red, serine green, tyrosine yellow) of homo- (dots) and hetero-peptides (circles); data for homo-peptides were taken from Piguet et al. 2018.

FIG. 9d shows, with respect to an embodiment of the invention: residence time scatter plots versus residual pore current I/Io with superimposed main level-averaged level histograms for the deca-peptides of conductor1 (red), conductor2 (blue), conductor3 (green), conductor4 (yellow), conductor5 (pink), conductor6 (black).

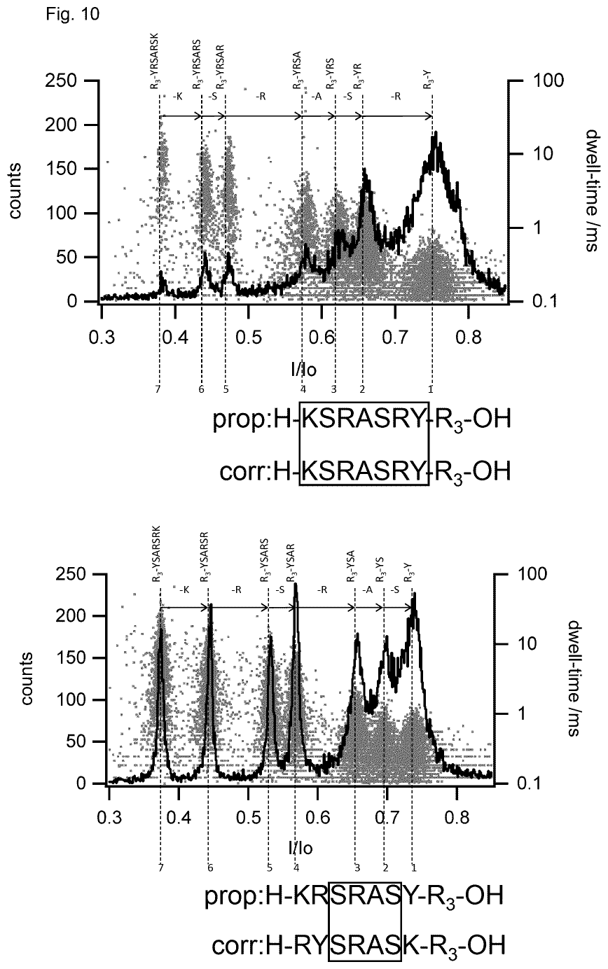

FIG. 10 shows, with reference to an embodiment of the invention: residence time scatter plots versus residual pore current I/Io (red) with superimposed level-averaged histograms (black) sample A (left) and B (right). Below each graph are the, using the first reader, proposed sequences (prop) and the correct sequences (corr). The green box indicates the correct reading frame.

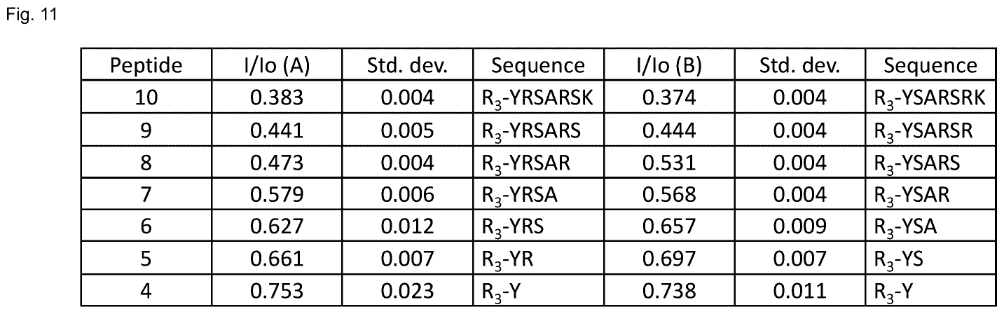

FIG. 11 shows in relation to an embodiment example of the invention: Data table for double-blind study.

FIG. 1a shows an illustration of the principle of single-molecule sensing through nanopores that can be used to implement the invention. A constant voltage □U across an insulator draws ionic current through the nanopore. A single analyte particle, e.g., a fragment, in the nanopore partially blocks the current (resistive pulse or current signal, or residual current value). Both the depth of the blockage and the duration carry information about the analyte.

FIG. 2 shows the two possible regimes of polymer-nanopore interaction. The threading/translocation regime is favored when long polyelectrolyte chains interact with the pore in low to moderate salt concentration (0.1 to 1.0 M KCl). The binding-trapping, or collapsed, regime typically occurs under conditions of high salt concentration (e.g., 4 M KCl) and does not require charging of the analyte. Preferably, the collapsed regime is used in the invention. In a measurement arrangement 1 for nanopore size spectroscopy, which can also be used in the method according to the invention, an electrolyte-filled first compartment 11 is electrically isolated from an electrolyte-filled second compartment 12 by a membrane formed, in particular, by means of a lipid bilayer 2; current flow is possible essentially only through the nanopore 3 incorporated in the lipid bilayer, which electrically connects the compartments 11 and 12. The lipid bilayer can be stretched over the microaperture or over a microcavity of a microstructure device (not shown in FIG. 2), as described, for example, in document WO 2013/083270. In the threading/translocation regime, the analyte 4a is elongated, and in the collapsed or binding regime, the analyte 4b is collapsed and compact.

FIG. 3 shows the detection of the twenty proteinogenic amino acids (aa) using the aerolysin nanopore.

A: 1: Peptide design 2: Peptide-pore interaction. 3: Current trace in the presence of a mixture of 7−R+D,K,R,E,H.

B: plot of relative current vs. aa volumes. C: >95% discrimination between structural isomers 7R+L and 7R+I by high-resolution recording on MECA (according to Ouldali et al. 2020).

Based on the prior art in Ouldali et al. 2020, the question for the inventors was how to use the high sensitivity of the nanopore to peptide size or volume for actual sequence identification in heteropolymers or for protein identification and sequencing.

To solve this problem, the inventors explored an approach, also called “nanopore ladder sequencing,” in which peptides (or other heteropolymers), which can be initially generated preferably by enzymatic or chemical or physical cleavage of proteins, are separated, preferably by known chromatographic or electrophoretic methods, or in which peptides or other heteropolymers are already present in isolation, and, preferably in a second step, are subjected either to the action of exopeptidases that cleave individual N- or C-terminal amino acids from a peptide, or to chemical methods such as the Edman reaction, in order to obtain a mixture of peptides or heteropolymers, i.e., a mixture of fragments, in which several species or characteristic fragment types are present in a representative set, preferably representing all or most of the possible fragments formed by the removal of amino acids (or monomer building blocks) in sequence, such that for a peptide (or heteropolymer) of degree of polymerization (d. p.) n, all or most species of d.p. n−(n−1), n−(n−2) . . . bis n(n−n) are present. Each of these species, when interacting with the nanopore, will give a characteristic maximum in the histogram of relative residual currents (characteristic residual current value or amount).

The measurement evidence demonstrates the ability of the invention here, for example, to correlate short, known peptide sequences with nanopore data in this manner (see FIG. 4). FIG. 4 shows:

A, B: Scatter plots with event histogram obtained from the interaction of aerolysin with two peptide ladders containing a triarginine handle. Removal of aa results in a species-specific shift in residual current characteristic of a monomer building block species (here aa).

C,D: Plot of the change in peptide volume and relative residual current for the two ladders shown above. A clear correlation between the two parameters as well as sequence dependence is evident.

FIG. 5a shows an exemplary method 100 according to the invention for identifying a sequence of monomer building blocks of a biological or synthetic heteropolymer, comprising the steps:
(a) carrying out a fragmentation method in which the heteropolymer is fragmented, in particular enzymatically, chemically and/or physically, and a fragment mixture is thereby obtained, the fragments of which are molecules having different sequence segments of the heteropolymer; (101)
(b) performing a current measurement method in which current signals of a current through a nanopore are detected, wherein each current signal is based on the interaction of a fragment with the nanopore, wherein the current signals are characteristic of the different fragments such that a representative set of characteristic current signals representing the fragment mixture is determinable; (102)
(c) Performing an evaluation method in which the sequence of the monomer building blocks of the heteropolymer is determined from the representative set of the characteristic current signals. (103)

In particular, the method 100 may be used in a method (200) for determining the primary structure of a protein, comprising the steps of (see FIG. 5b)
(i) cleavage of the protein, in particular by enzymatic and/or chemical and/or physical cleavage, to obtain peptides as cleavage products of the protein; optionally: obtaining the peptides by chromatographic or electrophoretic separation of a peptide mixture obtained by the cleavage; (201)
ii) Application of the method according to the invention for determining the sequence of amino acids (monomer building blocks) of at least one, in particular each, of the peptides (heteropolymer); (202 and 100, respectively).
(iii) performing a protein recognition procedure in which the primary structure of the protein is determined from the sequence of the at least one peptide. (203) For this purpose, in particular, method 100 may be carried out for all peptides obtained by cleavage of the protein.

The evaluation method (103 or 300), in which the sequence of the monomer building blocks of the heteropolymer is determined from the representative set of the characteristic current signals, may in particular comprise the following steps (see FIG. 5c):
A) determine residual current values from the measurement data, wherein a residual current describes the interaction of one of the different fragments of the heteropolymer with a nanopore; (301)
B) statistically determine a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence unambiguously, but in any case sufficiently for a desired structure elucidation or structure prediction; (302)
C) sort the characteristic residual current values by their magnitude to form a residual current value sequence and determining the current value differences of successive current values of the residual current value sequence; (303) and
(D) assign the current value differences to monomer building block species of the heteropolymer based on pre-known correlation data containing information about which monomer building block species is represented by which current value amount to perform the determination of the sequence of monomer building block species (determination of the sequence of monomer building blocks of the heteropolymer). (304)

Experimental Data and Embodiment

An embodiment of the invention is described below in which the complete sequence of synthetic peptides is elucidated, including in a double-blind experiment:

In the present embodiment, the method according to the invention is described as a “method for peptide sequence recognition with respect to peptide sequencing in a derivatization-free single molecule experiment using the wt-aerolysin (wt-AeL) nanopore by a bottom-up peptide ladder strategy”. In this research experiment, six peptide ladder-like sample pools were designed. Each pool consisted of the same deca-peptide but with a scrambled sequence and the respective ladder down to the polycationic tri-arginine carrier. Single molecule resistive pulse experiments (nanopore size spectroscopy) demonstrated the detection of species-dependent characteristic differences in residual current strengths for each peptide with identification of the single amino acid (aa) corresponding to each step of ladder formation, laying the foundation for peptide sequencing according to the invention. In addition, the potential of this simple approach as a benchmark technique in everyday laboratory use is described by a double-blind study in another laboratory in which two blindly selected peptides from the sample pool were identified and distinguished based on their aa sequence.

Peptide Ladder Design and Measurement

The embodiment uses the wt-AeL nanopore. A Deka peptide was designed consisting of a polycationic C-terminal carrier, R₃, preceded by a heterogeneous stretch of seven aa recruited from the five different aa SRAKY (e.g., SRASKYR). In a second step, the sequence of the aa portion was scrambled to obtain six different hetero-Deka peptides that have the exact same mass of 1335.65 Da (FIG. 6a). Next, peptide ladders (fragment mixtures) were formed for each Deka peptide down to R₃ (aa R₇₃, As R₆₃, . . . , aa R, R₁₃₃), resulting in a total of 42 samples. By successively adding the peptides of a ladder to the measurement chamber containing the nanopore, a stepwise degradation of a peptide in a ladder generation process was simulated (e.g., Edmann degradation). The step thus corresponds to step a) of the method according to the invention.

Step b) of the method according to the invention, or steps A) and B), was carried out as follows: In a typical experiment, a single wt-AeL channel was inserted into a DPhPC lipid bilayer spanning a single 50 μm aperture of the microelectrode cavity array (MECA16) used. A trans-negative bias voltage of 40 mV was used to drive an ion current (Io) through the protein channel connecting two reservoirs otherwise electrically isolated from each other by the lipid bilayer and filled with electrolyte solution (4 M KCl). Individual peptides that enter the channel defined by the protein and thereby alter the ionic current (I) are detected via the resulting resistive pulses, FIG. 6b. Ladder experiments were performed by adding all peptides of a ladder successively in equimolar amounts, starting with aa R₁₃ to aa R₇₃. FIG. 6e schematically shows a result of a nanopore-based peptide ladder experiment. The peptide ladder of an aa R₇₃ peptide would consist of eight peptides, each leading to a single maximum in the histogram of event-averaged residual current values. The sequence of maxima of the residual current histogram represents the sorting of the measured current signal values I as fractions of the current through the unblocked pore Io (also referred to as relative residual current values (I/Io) or relative residual conductances with possible values between 0 and 1) into a sequence of characteristic residual current values (step C)). It thus defines a representative set of 8 different characteristic residual current values with an equally characteristic dispersion, each representing a fragment of the peptide ladder. It is expected that the longest peptide, aa R₇₃, would lead to the deepest blockage, while the shortest peptide, R₃, would be represented with the highest I/Io. Then the sequence of maxima can also be clearly assigned to the steps of the ladder, and it is the difference in I/Io of two adjacent maxima that corresponds to the difference that the cleavage of a single aa would produce in the ladder generation process (used in step D). The magnitude of the difference ΔI/Io is thereby sensitive to the identity of the cleaved aa, which facilitates the identification of the sequence of the peptide.

An evaluation method in which the sequence of monomer building blocks (here: aa) of the heteropolymer (here: peptide) is determined from the representative set of characteristic current signals results from using the differences ΔI/Io of residual current values of adjacent maxima in the representative set of characteristic residual current values. Step D, determining the above aa, is performed by assigning the residual current value differences ΔI/Io to aa of the peptide using pre-known correlation data containing information about which aa is represented by which current value difference amount ΔI/Io to make the determination of the sequence of aa (determining the sequence of As of the peptide).

FIGS. 6c and d show exemplary raw data (current traces) for the measurement of the conductors L1. After addition of peptides (d), resistance pulses of different depth and duration were detected. It was seen that individual resistor pulses were strongly modulated, but to prevent distortion of the I/Io values, these modulations were excluded and only the main level of a pulse was considered in the data analysis. Such modulations are induced by the motion of the polymer itself within the AeL nanopore.

FIG. 6a: Sequences of the six heterodeca peptides, each representing the start peptide of a ladder. Black dashed boxes symbolize shifts of aa cassettes, black (and gray) lines symbolize inversion, while colored lines symbolize identity of aa in the different sequences; b: Schematic representation of the experimental setup. An external trans-negative voltage is applied to drive an ion current Io through the open nanopore. Peptides entering the nanopore alter the current, resulting in a resistive pulse (red curve); c: Control trace in 4 M KCl under a trans-negative voltage clamp of 40 mV, digitized at 1 MHz sampling rate, filtered with an 8-pole Bessel filter at a corner frequency of 50 kHz and digitally post-filtered at 25 kHz; d: Exemplary trace after addition of peptide ladder L1 with all peptides at equimolar concentration (H—SRASKYR—R₃ —OH, H—RASKYR—R₃ —OH, H—ASKYR—R₃ —OH, H—SKYR—R₃ —OH, H—KYR—R₃ —OH, H—YR—R₃ —OH, H—R—R₃ —OH); e: Schematic level histogram averaged over the main level for a peptide ladder sequencing experiment. The longest peptide (aa R₇₃) produces the deepest block, and the shortest peptide (aa R₁₃) produces the shallowest block. The differences in I/Io values (blue lines) can be correlated with the identity of the lost aa. The last aa can be determined against the polycationic C-terminal carrier peptide, R₃ (black).

To ensure correct assignment of maxima to peptides, the ladders were measured sequentially, starting with the smallest peptide. The expectation expressed above of a monotonic relationship between peptide length and depth of the block was confirmed. On this basis, following this experimental pathway, each of the 42 peptides could be identified within all six ladders (FIG. 7). Differences in the spacing of two adjacent maxima in the histograms are clearly visible and already indicate a presumed relationship between ΔI/Io and the identity of the cleaved aa. (Suppl. 1-Suppl. 6)

FIG. 7: Residence time scatter plots versus residual pore current I/Io (red) with superimposed histograms of relative residual current values averaged over the main resistive pulse current level (black) for all six peptide ladders. Peptides were added sequentially, starting with the smallest peptide aa R₁₃ and ending with the largest peptide aa R₇₃. All measurements of a ladder were performed using the same AeL nanopore. In addition, the green line indicates the location of the separately determined polycationic C-terminal carrier peptide, R₃.

All recorded resistive pulses in the data sets were analyzed in terms of event duration (dwell time) and amplitude (I/Io), as well as the number of modulations. The calculated differentials, i.e. changes in these values from one maximum to the next, were then plotted together with the differentials for the volume and hydrophobicity of the peptide against the respective position in the peptide, FIG. 8. To allow a direct comparison of all experiments, all differential values were double normalized with their maximum and minimum within the interval [0,1]. It was found that ΔI/Io correlated with the Δvolume (vol), indicating that the largest contribution to the blockade was caused by the volume of the analyte. Thus, the largest ΔI/Io was always found for arginine, the largest aa. Unexpectedly, serine always exhibited the smallest blockade, with one exception in L2, although the smallest volume change was expected for alanine. Remarkably, the ΔI/Io for uncharged and hydrophilic aa, tyrosine and serine, was always underweighted compared to their ΔVol, whereas hydrophobic alanine was found to be overweighted. On the other hand, charged aa, arginine and lysine, showed a different behavior. While arginine was found to be slightly overweighted in long peptides, it was found to be underweighted in short peptides. The opposite finding was found for lysine.

FIG. 8: Data correlation plots for all six peptide ladders. Dwell time scatter plots and level histograms averaged over the main level were analyzed for their differences in dwell time (red), residual current (blue), and number of modulations (black, dotted). The corresponding peptide volumes (green) and hydrophobicity (black, dashed) were also plotted. All values were double normalized to allow direct comparability.

Double-Blind Test

To investigate the reproducibility and reliability of the results described above, a double-blind experiment was performed. Six peptide ladder samples were prepared, each consisting of aa R₁₃ to aa R₇₃ in equimolar amounts. An independent third party acting as a notary randomly selected two of the six ladder samples, labeled them A & B, and sent them along with an R₃-homo peptide sample to an outside comparison laboratory (Abdelghani Oukhaled working group, Université Cergy Pontoise, France). In addition to the ladders, only FIG. 9b was initially submitted as a reading aid for the ladders, along with the information that all ladders consisted of a triarginine (R₃)C-terminus and the stoichiometric molecular formula A K R S₁₁₂₂₁, Yin every possible combination. In the comparative laboratory, the samples were analyzed under identical conditions but with different apparatus. Furthermore, the evaluation of the data, in particular the determination of the I/Io values, was carried out using our own algorithms and software routines, which differed significantly from those of the inventor's laboratory.

Using FIG. 9b alone, the sequence of sample A was correctly determined in the reference laboratory (KSRASRY, L3), and for sample B (FIG. 10) the partial sequence xxSRASx (i.e., more than half of the variable sequence components) was also correctly recognized and positioned here.

FIG. 10: Residence time scatter plots over the residual pore current I/Io (red) with superimposed level-averaged histograms (black) sample A (left) and B (right). Below each graph are the, using the first reader, proposed sequences (prop) and the correct sequences (corr). The green box indicates the correct reading frame.

### SUMMARY

The embodiment shows the method of the invention for peptide identification by ladder fingerprinting, which can serve as a primary platform for further development towards peptide sequencing, in particular using the highly sensitive wt-AeL nanopore. Reliable detection of hetero-peptides consisting of a c-terminal polycationic R₃-carrier and up to seven n-terminal alternating heterogeneous aa was achieved . . . . By using peptide ladder-like sample pools ranging from aa R₁₃ to aa R₇₃, the position-sensitive contribution of a specific aa species to the overall block depth of a peptide was investigated, and based on these findings, a sequencing as well as fingerprinting reading frame was postulated. Using these, the robustness and reliability of this strategy was demonstrated in a double-blind study by demonstrating sequencing of a randomly selected peptide and identification of a second peptide by fingerprinting.

In this embodiment example, peptides synthesized on demand were used. This is a model case that can be easily adapted for the case of unknown protein or peptide samples. More comprehensive analysis of larger heteropolymers is accomplished by an initial step of cleaving the heteropolymer by fragmentation methods into further fragmentable subcomponents, which are then used to form ladders For example, proteins can be made available in a standardized sample preparation process. Similar to standard bottom-up MS protein sequencing experiments, for example, an endo-peptidase can be used to fragment proteins into smaller peptides. Furthermore, an exo-peptidase can be used to dynamically generate ladders from these peptides. Individual peptides produced by the protease could be sequentially presented to the nanopore and analyzed in a dynamic exopeptidase-coupled experiment. There is great value in the method of the invention with respect to everyday laboratory applications.

Material and Methods

Reagents

All measurements were performed in AgCl (Carl Roth GmbH, Karlsruhe, Germany) saturated 4 M KCl (Carl Roth GmbH, Karlsruhe, Germany) buffered with 25 mM TRIS (Merck KGaA, Darmstadt, Germany) at pH 7.5. All solutions were prepared using 18.2 M Ω·cm⁻¹ Milli-Q water. After equilibration, the electrolyte solutions were filtered (0.22 μm) and stored protected from light. Peptides were synthesized according to the desired requirements by Intavis Peptide Services GmbH & Co KG (Tubingen, Germany). Stock solutions (750 μM) of all peptides were prepared in 10 mM HEPES, pH 7.5 and stored at −20° C. until use. Reagents were used at a final concentration of 5 μM.

Protein and Lipid Preparation

Wild-type proaerolysin (pAeL) was prepared internally via standard protocols from E. coli BL21 (DE3)-pLysS-competent cells using the pET22b (+) vector. pAeL was purified from cell lysates via His-tag chromatography. Sticks of pAeL were prepared using 1 μg·μL⁻¹, frozen with nitrogen, and stored at −80° C. Thawed pAeL was activated with trypsin (Promega GmbH, Walldorf, Germany) and used at a final pAeL concentration of 20 pmol L⁻¹ (or 3 pmol L⁻¹ AeL). The preprotein construct was chosen in such a way that the affinity tag used for purification is separated from the protein during trypsin activation and native protein is obtained.

All membranes were prepared from 1,2-diphytanoyl-sn-glycero-3-phosphocholine (DPhPC) from octane. DPhPC was dissolved in chloroform by Avanti Polar Lipids Inc (Alabaster, AL, USA). The lipids were aliquoted, dried under argon, and stored as a dry film at −20° C. until used at a concentration of 1 mg mL⁻¹

Nanopore Measurements Inventor Laboratory

All recordings were made using an Axopatch 200B (Molecular Devices, San Jose, CA, USA) in capacitive feedback mode with its 4-pole Bessel filter corner frequency set to 100 kHz at a digitization rate of 1 MHz. An 8-pole Bessel filter with a corner frequency of 50 kHz was connected between the amplifier output and the input of the analog-to-digital converter (Model 9002, Frequency Devices, Ottawa, II, USA). Digitization was performed using a National Instruments AD converter (PCI-6251, National Instruments, Austin, TX, USA). GePulse software (Michael Pusch, University of Genoa, Italy) was used for holding potential control and data recording. Single-molecule resistive pulses were collected under 40 mV transnegative voltage. To eliminate as many parasitic capacitances as possible, MECA16 cavity arrays from lonera GmbH (Freiburg, Germany) with 50 μm diameter cavities were used. Further digital filtering (25 kHz Bessel) and event detection was performed with self-written LabView (National Instruments)-based software; subsequent analysis with Igor Pro 8 (Wavemetrics, Lake Oswego, OR, USA).

Nanopore Measurements Comparison Lab:

All recordings were performed with an Axopatch 200B (Molecular Devices, San Jose, CA, USA) in resistive feedback mode with its 4-pole Bessel filter cutoff frequency set to 5 kHz at a digitization rate of 100 kHz. A classic vertical chamber system from Warner Instruments (Hamden, CT, USA) with apertures of 150 μm diameter was used for the measurements. Digitization was performed using the DigiDatat 1440A AD converter and Clampex10 software (Molecular Devices). The analysis was performed with in-house routines implemented in IgorPro 8.

Suppl. 1 (Supplement 1): determined values from peptide ladder L1
Ladder L₁

| | | | | | | | norm Δ | | | |
|---|---|---|---|---|---|---|---|---|---|---|
| | loss | | | norm | dwell- | Δ dwell | dwell- | | Δ | norm Δ |
| sequence | of | I/lo | ΔI/lo | ΔI/lo | time/ms | time/ms | time | n_m2 | dn_m2 | dn_m2 |
| SRASK | | 0.3686 | — | — | 9.073 | — | — | 3.35 | — | — |
| YR-R₃ | | | | | | | | | | |
| RASK | S | 0.3922 | 0.0235 | 0.0000 | 10.419 | −1.346 | 0.000 | 3.07 | 0.29 | 0.35 |
| YR-R₃ | | | | | | | | | | |
| ASK YR-R₃ | R | 0.4965 | 0.1044 | 1.0000 | 3.909 | 6.510 | 1.000 | 2.55 | 0.52 | 0.645 |
| SK YR-R₃ | A | 0.5360 | 0.0395 | 0.1975 | 2.412 | 1.497 | 0.361 | 1.75 | 0.80 | 1.00 |
| K YR-R₃ | S | 0.5622 | 0.0262 | 0.0329 | 2.034 | 0.379 | 0.220 | 1.59 | 0.16 | 0.19 |
| YR-R₃ | K | 0.6487 | 0.0865 | 0.7782 | 0.690 | 1.344 | 0.342 | 1.14 | 0.46 | 0.57 |
| R-R₃ | Y | 0.7259 | 0.0772 | 0.6642 | 0.167 | 0.523 | 0.238 | 1.01 | 0.13 | 0.15 |
| R₃ | R | 0.8067 | 0.0809 | 0.7089 | 0.021 | 0.146 | 0.190 | 1.00 | 0.01 | 0.00 |

Suppl. 2 (Supplement 2): determined values from peptide ladder L2
Ladder L₂

| | | | | | | | norm Δ | | | |
|---|---|---|---|---|---|---|---|---|---|---|
| | loss | | | norm | dwell- | Δ dwell | dwell- | | Δ | norm Δ |
| sequence | of | I/lo | ΔI/lo | ΔI/lo | time/ms | time/ms | time | n_m2 | dn_m2 | dn_m2 |
| KSRYA | | 0.3792 | — | — | 4.952 | — | — | 4.03 | — | — |
| RS-R₃ | | | | | | | | | | |
| SRYA | K | 0.4418 | 0.0625 | 0.4837 | 2.120 | 2.832 | 1.000 | 1.90 | 2.14 | 1.00 |
| RS-R₃ | | | | | | | | | | |
| RYA | S | 0.4837 | 0.0419 | 0.0993 | 1.891 | 0.229 | 0.076 | 1.68 | 0.22 | 0.10 |
| RS-R₃ | | | | | | | | | | |
| YA RS-R₃ | R | 0.5739 | 0.0902 | 1.0000 | 0.694 | 1.198 | 0.420 | 1.22 | 0.46 | 0.22 |
| A RS-R₃ | Y | 0.6481 | 0.0742 | 0.7003 | 0.233 | 0.460 | 0.158 | 1.03 | 0.19 | 0.09 |
| RS-R₃ | A | 0.6846 | 0.0366 | 0.0000 | 0.164 | 0.070 | 0.020 | 1.02 | 0.01 | 0.00 |
| S-R₃ | R | 0.7603 | 0.0756 | 0.7279 | 0.035 | 0.128 | 0.040 | 1.00 | 0.02 | 0.01 |
| R₃ | S | 0.8067 | 0.0465 | 0.1848 | 0.021 | 0.014 | 0.000 | 1.00 | 0.00 | 0.00 |

Suppl. 3 (Supplement 3): values determined from peptide ladder L3
Ladder L₃

| | | | | | | | norm Δ | | | |
|---|---|---|---|---|---|---|---|---|---|---|
| | loss | | | norm | dwell- | Δ dwell | dwell- | | Δ | norm Δ |
| sequence | of | I/lo | ΔI/lo | ΔI/lo | time/ms | time/ms | time | n_m2 | dn_m2 | dn_m2 |
| KSRAS | | 0.3869 | — | — | 4.082 | — | — | 3.05 | — | — |
| RY-R₃ | | | | | | | | | | |
| SRAS | K | 0.4444 | 0.0575 | 0.3533 | 2.695 | 1.387 | 0.72128 | 1.99 | 1.06 | 1.00 |
| RY-R₃ | | | | | | | | | | |
| RAS | S | 0.4749 | 0.0305 | 0.0000 | 2.847 | −0.152 | 0.000 | 1.98 | 0.01 | 0.00 |
| RY-R₃ | | | | | | | | | | |
| AS RY-R₃ | R | 0.5819 | 0.1069 | 1.0000 | 0.865 | 1.982 | 1.000 | 1.39 | 0.60 | 0.56 |
| S RY-R₃ | A | 0.6233 | 0.0414 | 0.1424 | 0.479 | 0.385 | 0.252 | 1.13 | 0.25 | 0.23 |
| RY-R₃ | S | 0.6564 | 0.0331 | 0.0331 | 0.417 | 0.063 | 0.101 | 1.09 | 0.04 | 0.03 |
| Y-R₃ | R | 0.7442 | 0.0878 | 0.7497 | 0.105 | 0.312 | 0.218 | 1.01 | 0.08 | 0.07 |
| R₃ | Y | 0.8067 | 0.0626 | 0.4191 | 0.021 | 0.084 | 0.111 | 1.00 | 0.01 | 0.00 |

Suppl. 4 (supplement 4): determined values from peptide ladder L4
Ladder L₄

| | | | | | | | norm Δ | | | |
|---|---|---|---|---|---|---|---|---|---|---|
| | loss | | | norm | dwell- | Δ dwell | dwell- | | Δ | norm Δ |
| sequence | of | I/lo | ΔI/lo | ΔI/lo | time/ms | time/ms | time | n_m2 | dn_m2 | dn_m2 |
| RYSRA | | 0.3627 | — | — | 4.173 | — | — | 1.72 | — | — |
| SK-R₃ | | | | | | | | | | |
| YSRA | R | 0.4372 | 0.0745 | 0.7394 | 2.608 | 1.565 | 1.000 | 1.52 | 0.20 | 0.59 |
| SK-R₃ | | | | | | | | | | |
| SRA SK-R₃ | Y | 0.5226 | 0.0854 | 0.9493 | 1.432 | 1.126 | 0.717 | 1.18 | 0.34 | 1.00 |
| RA SK-R₃ | S | 0.5585 | 0.0359 | 0.0000 | 1.052 | 0.430 | 0.269 | 1.08 | 0.09 | 0.27 |
| A SK-R₃ | R | 0.6465 | 0.0880 | 1.0000 | 0.270 | 0.782 | 0.496 | 1.01 | 0.07 | 0.21 |
| SK-R₃ | A | 0.6863 | 0.0398 | 0.0745 | 0.142 | 0.128 | 0.074 | 1.01 | 0.00 | 0.01 |
| K-R₃ | S | 0.7307 | 0.0444 | 0.1629 | 0.130 | 0.012 | 0.000 | 1.00 | 0.01 | 0.02 |
| R₃ | K | 0.8067 | 0.0760 | 0.7695 | 0.021 | 0.109 | 0.062 | 1.00 | 0.00 | 0.00 |

Suppl. 5 (supplement 5): determined values from peptide ladder L5
Ladder L₅

| | | | | | | | norm Δ | | | |
|---|---|---|---|---|---|---|---|---|---|---|
| | loss | | | norm | dwell- | Δ dwell | dwell- | | Δ | norm Δ |
| sequence | of | I/lo | ΔI/lo | ΔI/lo | time/ms | time/ms | time | n_m2 | dn_m2 | dn_m2 |
| KRSSR | | 0.3793 | — | — | 3.514 | — | — | 2.35 | — | — |
| AY-R₃ | | | | | | | | | | |
| RSSR | K | 0.4404 | 0.0611 | 0.3874 | 2.353 | 1.161 | 0.732 | 1.86 | 0.48 | 0.95 |
| AY-R₃ | | | | | | | | | | |
| SSR | R | 0.5352 | 0.0948 | 1.0000 | 0.783 | 1.570 | 1.000 | 1.35 | 0.51 | 1.00 |
| AY-R₃ | | | | | | | | | | |
| SR | S | 0.5780 | 0.0428 | 0.0548 | 0.666 | 0.116 | 0.046 | 1.24 | 0.12 | 0.23 |
| AY-R₃ | | | | | | | | | | |
| R AY-R₃ | S | 0.6178 | 0.0398 | 0.0000 | 0.616 | 0.051 | 0.003 | 1.14 | 0.10 | 0.19 |
| AY-R₃ | R | 0.6968 | 0.0790 | 0.7127 | 0.147 | 0.468 | 0.277 | 1.02 | 0.13 | 0.24 |
| Y-R₃ | A | 0.7435 | 0.0468 | 0.1263 | 0.101 | 0.046 | 0.000 | 1.00 | 0.01 | 0.02 |
| R₃ | Y | 0.8067 | 0.0632 | 0.4262 | 0.021 | 0.080 | 0.023 | 1.00 | 0.00 | 0.00 |

Suppl. 6 (supplement 6): determined values from peptide ladder L6
Ladder L₆

| | | | | | | | norm Δ | | | |
|---|---|---|---|---|---|---|---|---|---|---|
| | loss | | | norm | dwell- | Δ dwell | dwell- | | Δ | norm Δ |
| sequence | of | I/lo | ΔI/lo | ΔI/lo | time/ms | time/ms | time | n_m2 | dn_m2 | dn_m2 |
| SKRYS | | 0.3937 | — | — | 4.738 | — | — | 2.28 | — | — |
| RA-R₃ | | | | | | | | | | |
| KRYS | S | 0.4179 | 0.0242 | 0.0000 | 4.811 | −0.073 | 0.000 | 2.11 | 0.17 | 0.32 |
| RA-R₃ | | | | | | | | | | |
| RYS | K | 0.4901 | 0.0722 | 0.7117 | 2.087 | 2.723 | 1.000 | 1.58 | 0.53 | 1.00 |
| RA-R₃ | | | | | | | | | | |
| YS RA-R₃ | R | 0.5817 | 0.0916 | 1.0000 | 0.712 | 1.376 | 0.518 | 1.24 | 0.34 | 0.65 |
| S RA-R₃ | Y | 0.6601 | 0.0784 | 0.8047 | 0.268 | 0.443 | 0.185 | 1.02 | 0.22 | 0.42 |
| RA-R₃ | S | 0.6919 | 0.0318 | 0.1129 | 0.218 | 0.051 | 0.044 | 1.01 | 0.01 | 0.02 |
| A-R₃ | R | 0.7627 | 0.0708 | 0.6917 | 0.050 | 0.167 | 0.086 | 1.00 | 0.01 | 0.01 |
| R₃ | A | 0.8067 | 0.0441 | 0.2950 | 0.021 | 0.029 | 0.037 | 1.00 | 0.00 | 0.00 |

Suppl. 7 (Supplement 7): determined values for I/lo and residence time of homo-arginine
peptides. Ensslen et al. Refers to the embodiment according to the invention.

| | Piguet et al. (−50 mV) | | | | Ensslen et al. (−40 mV) | |
|---|---|---|---|---|---|---|
| Rx | I/lo | ΔI/lo | dwell-time/ms | Δdwell-time/ms | I/lo | dwell-time/ms |
| 10 | 0.234 | — | 72.0 | — | — | — |
| 9 | 0.286 | 0.052 | 31.0 | 41.0 | — | — |
| 8 | 0.353 | 0.067 | 14.2 | 16.8 | — | — |
| 7 | 0.435 | 0.082 | 6.2 | 8.0 | 0.4371 | 7.23 |
| 6 | 0.530 | 0.095 | 2.3 | 3.9 | — | — |
| 5 | 0.631 | 0.101 | 0.9 | 1.4 | 0.6309 | 0.86 |
| 4 | 0.731 | 0.1 | — | — | 0.7259 | 0.167 |
| 3 | — | — | — | — | 0.8067 | 0.02 |

1. A method for identifying a sequence of monomer building blocks of a biological or synthetic heteropolymer, comprising the steps:
a) perform a fragmentation method in which the heteropolymer is broken down into fragments, thereby obtaining a fragment mixture whose fragments are molecules having different sequence segments of the heteropolymer;
b) perform a current measurement method in which current signals of a current through the channel of a nanopore are detected, wherein each current signal is based on the interaction of a fragment of the fragment mixture with the channel of the nanopore, wherein the current signals are characteristic of the different fragments such that a representative set of characteristic current signals representing the fragment mixture is determinable; and
c) perform an evaluation method in which a sequence of monomer building blocks of the heteropolymer is determined from the representative set of characteristic current signals.

2. The method according to claim 1, wherein the fragments of the fragment mixture are obtained by enzymatic, chemical and/or physical methods and/or are obtained by successive degradation of the heteropolymer.

3. The method according to claim 2, wherein the successive degradation of the heteropolymer provides that the heteropolymer is chain-like and, starting from one end of its chain, is stepwise shortened by one monomer building block to obtain length fragments, in particular substantially all length fragments n-(n-1), n-(n-2) . . . to n−(n−n), of a heteropolymer consisting of n monomer building blocks.

4. The method according to claim 1, wherein the heteropolymer is a peptide and the fragmentation method is or includes Edman degradation.

5. A method according to claim 1, for determining the primary structure of a macromolecule formed at least from heteropolymers, in particular a protein, comprising the steps of:
i) cleavage of the macromolecule, in particular by enzymatic and/or chemical and/or physical cleavage, to obtain heteropolymers, in particular peptides, as cleavage products of the macromolecule; optionally: obtaining the heteropolymers by chromatographic or electrophoretic separation of a heteropolymer mixture obtained by the cleavage;
ii) use of the method according to claim 1 for determining a sequence of monomer building blocks, in particular amino acids, of at least one, in particular each, of the heteropolymers;
iii) perform a macromolecule recognition method in which the primary structure of the macromolecule is determined from a sequence listing of the at least one heteropolymer.

6. The method according to claim 5, wherein the macromolecule is DNA, RNA, protein, peptide, or any synthetic polymer, and wherein, in particular, the nanopore is a biological nanopore or a toxin or pore-forming toxin.

7. The method according to claim 1, wherein the nanopore is a solid-state nanopore or a hybrid of solid-state and biological components.

8. The method according to claim 1, wherein the fragmentation of the heteropolymer is carried out by enzymes.

9. The method according to claim 1, wherein the fragmentation of the heteropolymer is carried out chemically and non-enzymatically.

10. The method according to claim 1, wherein the fragmentation of the heteropolymer is carried out physically, e.g. by exposure to heat, cold, sound waves, electromagnetic radiation, in particular infrared, ultraviolet or X-ray radiation, microwaves or visible light.

11. The method according to claim 1, wherein the nanopore is aerolysin, alpha-hemolysin, VDAC, or other protein of the beta-barrel protein family.

12. Use of a nanopore for performing the method for identifying a sequence of monomer building blocks of a biological or synthetic heteropolymer according to claim 1.

13. A computer-implemented method for determining a sequence of monomer building blocks of a heteropolymer, referred to as a heteropolymer sequence, from measurement data of a current measurement method containing information on current signals obtained upon interaction of different fragments formed from the heteropolymer with the channel of a nanopore, comprising the steps of:
A) determine residual current values from the measurement data, wherein a residual current describes the interaction of one of the different fragments of the heteropolymer with the channel of a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set uniquely describing the heteropolymer sequence;
C) sort the characteristic residual current values by their magnitude into a residual current value sequence and determining the current value differences of successive current values of the residual current value sequence; and
D) assign the current value differences to monomer building block types of the heteropolymer based on previously known correlation data containing information about which monomer building block type is represented by which current value amount to make the determination of the sequence of monomer building block types.

14. A computer program code which is stored on a data carrier and which determines a sequence of monomer building blocks of a heteropolymer, referred to as heteropolymer sequence, from the measurement data of a current measurement method when executed by the central processor of a computer, the measurement data containing information on current signals which are determined upon the interaction of different fragments formed from the heteropolymer with a nanopore, comprising the respective steps implemented by program code:
A) determine residual current values from the measurement data, wherein a residual current describes the interaction of one of the different fragments of the heteropolymer with a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence unambiguously, but in any case sufficiently for a desired structure elucidation or structure prediction;
C) sort the characteristic residual current values by their magnitude into a residual current value sequence and determining the current value differences of successive current values of the residual current value sequence; and
D) assign the current value differences to monomer building block types of the heteropolymer based on previously known correlation data containing information about which monomer building block type is represented by which current value amount to make the determination of the sequence of monomer building block types.

15. A data processing system for determining a sequence of monomer building blocks of a heteropolymer, referred to as heteropolymer sequence, from the measurement data of a current measurement method containing information on current signals determined upon interaction of different fragments formed from the heteropolymer with a nanopore, comprising a computer with a central processor, and a program code, in particular the program code according to claim 14, wherein the computer is programmed to perform the following computer-implemented steps:
A) determine residual current values from the measured data, wherein a residual current describes the interaction of one of the different fragments of the heteropolymer with a nanopore;
B) statistically determine of a representative set of characteristic residual current values from the residual current values, a characteristic residual current value describing in each case one fragment type, in particular fragment size, of the number n of fragment types of a fragment mixture formed from the heteropolymer, the representative set describing the heteropolymer sequence unambiguously, but in any case sufficiently for a desired structure elucidation or structure prediction;
C) sort the characteristic residual current values according to their contribution to a residual current value sequence and determine the current value differences of successive current values of the residual current value sequence; and
D) assign the current value differences to monomer building block types of the heteropolymer based on pre-known correlation data containing information about which monomer building block type is represented by which current value amount to perform the determination of the sequence of monomer building block types.
